(19) United States
(12) Patent Application Publication
(10) Pub. No.: US 2014/0371781 A1
(43) Pub. Date: Dec. 18, 2014

(54) COVERED FILTER CATHETER APPARATUS AND METHOD OF USING SAME

(71) Applicant: BIO2 MEDICAL, INC., San Antonio, TX (US)

(72) Inventor: Jeremy Morgan, Idaho Springs, CO (US)

(21) Appl. No.: 14/307,406

(22) Filed: Jun. 17, 2014

Related U.S. Application Data
(60) Provisional application No. 61/836,069, filed on Jun. 17, 2013.

(51) Int. Cl. A61F 2/01; A61L 31/14

(52) U.S. Cl. CPC A61F 2/01; A61L 31/146; A61F 2002/011; A61F 2210/0014; A61F 2230/006; A61F 2230/0086; A61F 2210/0071; A61F 2230/0069
USPC 606/200

(57) ABSTRACT

A combined catheter and an embolic filter, further including a porous section coupled to at least a distal portion of the filter, wherein the porous section comprises a cover member may comprise high porosity ePTFE, or a netting forming a soft basket.

### CROSS-REFERENCE TO RELATED APPLICATIONS

This non-provisional application claims the benefit of the priority of U.S. Provisional Application Ser. No. 61/836,069, which was filed on Jun. 17, 2013, and is incorporated by reference herein in its entirety.

### BACKGROUND OF THE INVENTION

The present invention relates generally to embolic filter devices for placement in the vasculature, and in particular to self-expanding frames used to support embolic filter elements.

Embolic protection is a concept of growing clinical importance directed at reducing the risk of embolic complications associated with interventional (i.e., transcatheter) and surgical procedures. In therapeutic vascular procedures, liberation of embolic debris (e.g., thrombus, clot, atheromatous plaque, etc.) can obstruct perfusion of the downstream vasculature, resulting in cellular ischemia and/or death. The therapeutic vascular procedures most commonly associated with adverse embolic complications include: carotid angioplasty with or without adjunctive stent placement; and revascularization of degenerated saphenous vein grafts. Additionally, percutaneous transluminal coronary angioplasty with or without adjunctive stent placement, surgical coronary artery by-pass grafting, percutaneous renal artery revascularization, and endovascular aortic aneurysm repair have also been associated with complications attributable to atheromatous embolization. The use of embolic protection devices to capture and remove embolic debris, consequently, may improve patient outcomes by reducing the incidence of embolic complications.

The placement of embolic protection devices typically occurs concomitantly with central access line placement or in critically ill patients that already have a central access line in place. The more recent development of devices that combine the function of a central access catheter and a removable embolic protection device, or filter, will streamline the process of deployment and retrieval of temporary filters. Examples of such devices are disclosed in U.S. Pat. Nos. 8,613,753 and 8,668,712, the contents of which are incorporated by reference herein. These filters and the other similar retrievable filters are made of bio-compatible metal, or metal-like materials, typically made to expand and engage the blood vessel wall once deployed. The filters tend to be coarse or even rigid once deployed as it is critical that the perimeter of the opening of the filter be in contact with the blood vessel wall to minimize the potential for thrombic material bypass the filter opening. Additionally, the design of these filters results in increased contact with the vessel wall as the vessel diameter decreases in size, increasing risk of injury to the vessel wall.

There is a need in the art for filters that minimize the risk of injury to the blood vessel wall but yet is capable of engaging with the wall of the blood vessel to ensure that thrombic material does not bypass the filter.

### SUMMARY OF THE INVENTION

In view of the above, filters are provided for trapping thrombi in a blood vessel while minimizing the risk of injury to the blood vessel wall. In an example implementation, a filter comprises a frame formed by a plurality of frame members extending from a frame end to a plurality of basket fixation elements. A soft basket comprised of netting surrounding an inner basket region is attached to the frame using a plurality of basket attachment portions attached to corresponding basket fixation elements. The inner basket region of the netting is closed at a distal end by a distal basket closure.

In another aspect of the invention, a method is provided for capturing thrombi in a blood vessel. In an example method, a catheter having a filter attached to a distal end of the catheter is introduced into the blood vessel. The filter comprises a frame formed by a plurality of frame members and a soft basket attached to the plurality of frame members. The soft basket has an opening at the attachment to the frame and a distal basket closure. The filter is deployed within the blood vessel by moving the frame to a region of interest to permit expansion of the frame within the blood vessel and to permit expansion of the soft basket. The soft basket expands so that the opening of the soft basket faces a patient's blood flow and the distal basket closure is pushed distally by the patient's blood flow.

Various advantages, aspects and novel features of the present invention, as well as details of an illustrated embodiment thereof, will be more fully understood from the following description and drawings.

Other systems, methods and features of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

FIG. 1 is a perspective view of a filter catheter in accordance with a first embodiment of the present invention with the filter in an unexpanded state.

FIG. 2 is a side elevational view of a filter catheter in accordance with the first embodiment of the present invention.

FIG. 3. is a cross-sectional view taken along line 3-3 of FIG. 2.

FIG. 4 is a cross-sectional view taken along line 4-4 of FIG. 2.

FIG. 5 is a cross-sectional view taken along line 5-5 of FIG. 2.

FIG. 6 is a perspective view of a filter catheter in accordance with a second embodiment of the present invention illustrating the filter in an unexpanded state.

FIG. 7 is a side elevational view of a filter catheter in accordance with the second embodiment of the present invention.

FIG. 8 is a cross-sectional view taken along line 8-8 of FIG. 7.

FIG. 9 is a cross-sectional view taken along line 9-9 of FIG. 7.

FIG. 10 is a cross-sectional view taken along line 10-10 of FIG. 7.

FIG. 11 is a cross-sectional view taken along line 11-11 of FIG. 7.

FIG. 12A is a perspective view of the filter catheter of FIG. 1 illustrating the filter in a diametrically expanded state; and FIG. 12B is a perspective view of the filter catheter of FIG. 12A, further comprising a high porosity ePTFE cover member.

FIG. 13A is a perspective view of a filter member in accordance with a first embodiment thereof.

FIG. 13B is a first side elevational view thereof.

FIG. 13C is an end elevational view thereof.

FIG. 13D is a second side elevational view thereof.

FIG. 13E is a perspective view of the filter member of FIG. 13A, further comprising a high porosity ePTFE cover member; FIG. 13F is a first side elevational view thereof; FIG. 13G is an end elevational view thereof; and FIG. 13H is a second side elevational view thereof.

FIGS. 14A-14H are perspective views of alternative embodiments of a filter member in accordance with the present invention.

FIG. 15A-15H are fragmentary side elevational views of the alternative embodiments of the filter member illustrated in FIGS. 14A-14H.

FIG. 16A is a side elevational view of the filter catheter in its undeployed state.

FIG. 16B is a side elevational view of the filter catheter in its deployed state.

FIG. 17A is a side elevational view of a filter member in its expanded state in accordance with one embodiment of the present invention; and FIG. 17B is a side elevational view of the filter member of FIG. 17A further comprising a high porosity ePTFE cover member.

FIG. 18A is a perspective view of a filter member in its expanded state in accordance with an alternative embodiment of the present invention; and FIG. 18B is a perspective view of the filter member of FIG. 18A further comprising a high porosity ePTFE cover member.

FIG. 19A is a perspective view of a filter member in its expanded state in accordance with yet another embodiment of the present invention; and FIG. 19B is a perspective view of the filter member of FIG. 19A further comprising a high porosity ePTFE cover member.

FIG. 20A is a perspective view of a filter member in its expanded state in accordance with still another embodiment of the present invention; and FIG. 20B is a perspective view of the filter member of FIG. 20A further comprising a high porosity ePTFE cover member.

FIGS. 21A and 21B are perspective views of a filter member mounted at a distal end of a filter catheter having a distal balloon.

FIGS. 22A and 22B are perspective views of an alternative embodiment of a filter member mounted at a distal end of a filter catheter having a distal balloon.

FIG. 23 is a side view of an example of a filter having a soft basket.

FIG. 24 is a perspective view of another example of a filter having a soft basket.

FIG. 25 is a side cross-sectional view of an example of a basket attachment tube element.

FIG. 26 is a side view of another example of a basket attachment tube element with a fixation portion.

FIG. 27 is a side view of another example of a basket attachment tube element with a loop.

FIG. 28A is a top view of a section of an example implementation of the netting for a soft basket.

FIG. 28B shows another example implementation of the netting for a soft basket.

FIG. 28C shows another example implementation of the netting for a soft basket.

FIGS. 29A and 29B illustrate an example implementation of a frame and basket fixation elements that close the soft basket portion of the filter by pulling on the frame.

FIGS. 30A and 30B illustrate deployment of an example filter with a soft basket using a balloon catheter.

FIGS. 31A through 31C illustrate deployment of an example filter with a soft basket using a frame deployment member and a basket deployment member.

### DETAILED DESCRIPTION OF THE PREFERRED EMBODIMENTS

The present invention, in one embodiment, provides a device adapted for deployment in a body vessel for collecting emboli. The device includes a filter member coupled to a catheter or guidewire for insertion. In some embodiments, the filter may further include a cover member to provide enhanced filtering. In some embodiments, the cover member includes a high porosity as to permit fluid flow. In other embodiments, the cover member may comprise other biocompatible materials as discussed below. Alternatively, the cover member may provide an occlusion capability to the device. The cover member may be disposed over a distal portion of the filter member. The filter member is sized to extend to walls of a body cavity in an expanded deployed profile for collecting emboli floating in the body cavity.

In an example embodiment, the filter member comprises a frame and a soft basket connected to the frame. The frame provides a structure for expanding and maintaining the soft basket open to capture emboli. The frame may also provide a deployment mechanism and in another example implementation, a mechanism for closing the soft basket.

One aspect of the present invention is to provide a filter geometry in which the proximal portion of the filter, relative to the axis of blood flow, has larger interstitial openings to permit thrombus or embolic material to flow into the filter, while the distal portion of the filter, again relative to the axis of blood flow, has relatively smaller interstitial openings that capture the thrombus or embolic material within the filter. Another way to view this aspect is that the structure of the filter includes a greater open surface area exposed to the flow of embolic material into the filter at its proximal end, while the distal end has smaller open surface area exposed to the flow of embolic material to capture the embolic material in the distal end of the filter member.

In some embodiments, a drug or other biologically active compound may be loaded into the pores of the high porosity cover member, and eluted therefrom after deployment of the filter member.

The device may be configured as a distal component of a central access catheter.

Alternatively the device may comprise a distal component of a peripherally inserted central catheter (PICC). A PICC is a form of intravenous access that can be used for a prolonged period of time (e.g. for long chemotherapy regimens, extended antibiotic therapy, or total parenteral nutrition). A PICC is an alternative to subclavian lines, internal jugular lines or femoral lines which have higher rates of infection. A PICC is inserted in a peripheral vein, such as the cephalic vein, basilic vein, or brachial vein and then advanced through increasingly larger veins, toward the heart until the tip rests in the distal superior vena cava or cavoatrial junction. The insertable portion of a PICC varies from 25 to 60 cm in length, that being adequate to reach the desired tip position in most patients. Some lines are designed to be trimmed to the desired length before insertion; others are simply inserted to the needed depth with the excess left outside. As supplied, the line may include a guide wire inside, which is provided to stiffen the (otherwise very flexible) line so it can be threaded through the veins.

The present invention relates generally to access catheters having a filter at a distal end. Implementations of the catheter may also include a port proximal the filter, a port distal the filter and plural infusion ports. The proximal and distal ports permit measuring pressure and/or flow velocity across the filter as a determinant of extent of capture of embolic material in the filter or measuring flow rate at the position of the filter member as a positional indicator within the body. The proximal and distal ports also provide means for introducing a bioactive agent, such as an anticoagulant or thrombolytic agents, contrast medium, blood transfusions, fluids or medications. The multiple infusion ports also provide a means for introducing a flushing medium, such as saline, under elevated pressure to produce mechanical thrombolysis or induce thrombolysis by the infusion of thrombolytic agents directly to thrombus within the filter. The filter may be covered with a high porosity ePTFE cover member to provide enhanced filtering or to provide an occlusion capability.

Accordingly, in one embodiment, it is an objective of the present invention to provide a multi-lumen catheter coupled to a vena cava filter that is useful both as a central venous access catheter for administration of intravenous fluids, bioactive agents, contrast agents, flushing agents, pressurized fluids for mechanical thrombolysis and/or withdrawal of blood samples and for capture of thrombus or emboli.

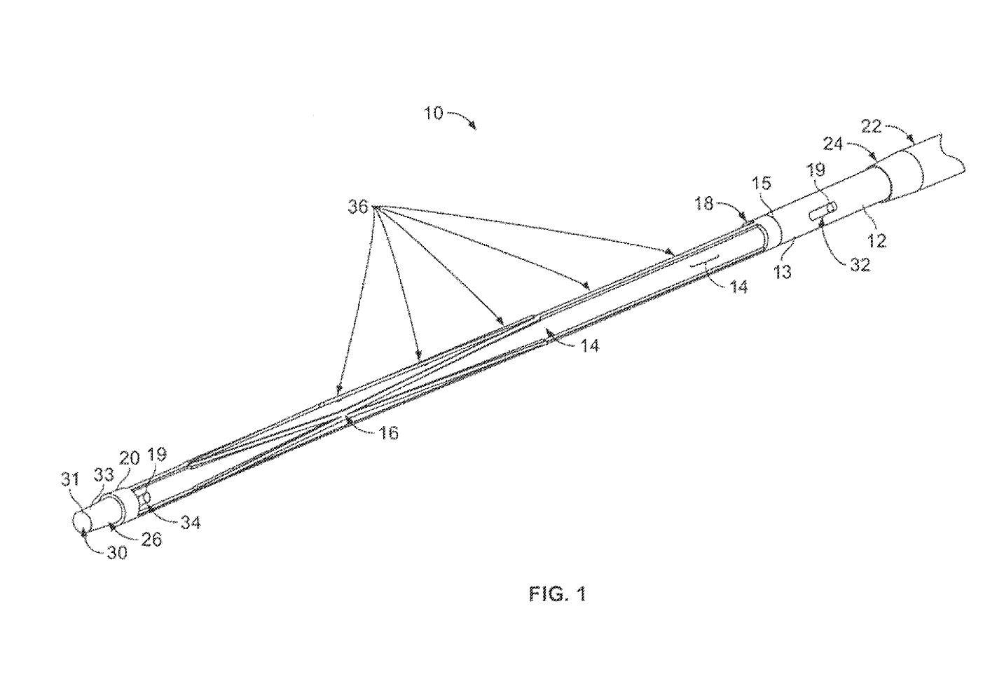

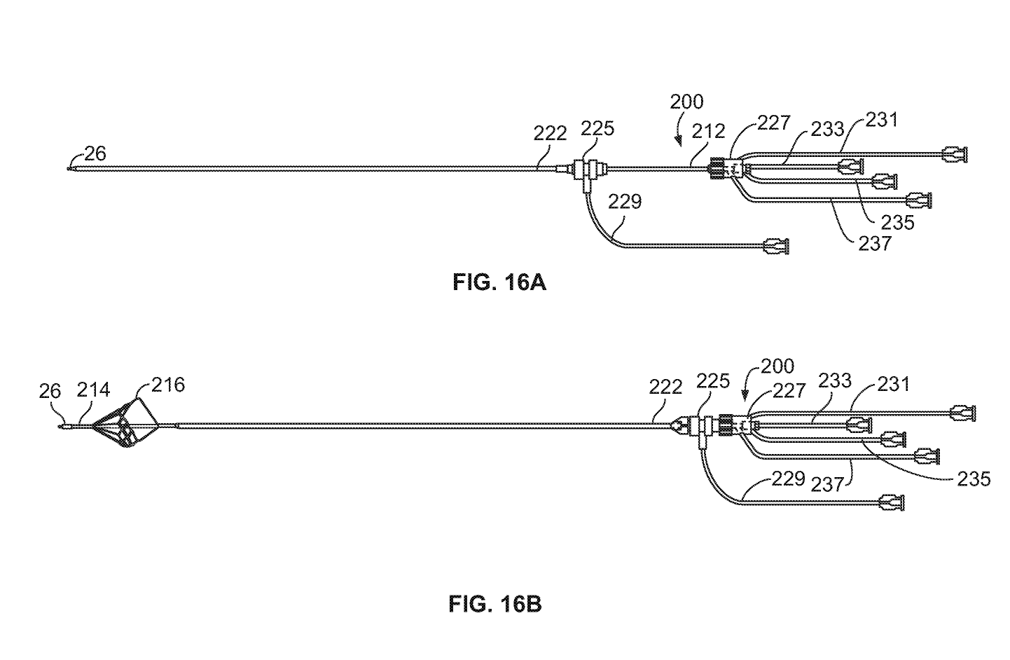

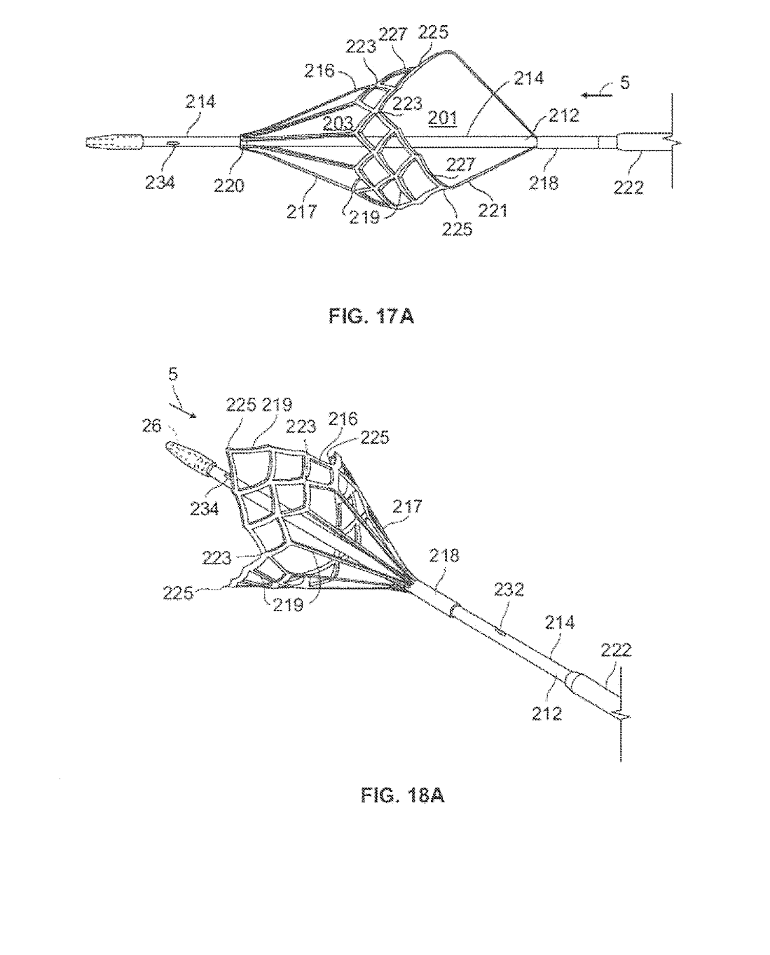

In the accompanying Figures like structural or functional elements are designated by like reference numerals, e.g., 16, 116, 216, 316, 416 represent similar structural or functional elements across different embodiments of the invention. With particular reference to FIGS. 1-5, according to a first embodiment of the invention, there is disclosed a filter catheter 10 that is composed generally of a multi-lumen central catheter body 12 having a proximal port 32 associated with a first lumen 44 and a distal port 34 associated with a second lumen 42. A filter member 16, having a first end 18 and a second end 20, is positioned generally intermediate the distal port 34 and the proximal port 32 and is generally concentric relative to the catheter body 12. An outer sheath 22 may be concentrically disposed over the catheter body 12 such that relative movement of the catheter body 12 and the outer sheath 22 either exposes the filter member 16 or captures the filter member 16 within the outer sheath 22. The outer sheath 22 terminates in an annular opening at a distal end thereof and at first hub member 225 as depicted in FIGS. 16A and 16B. The proximal hub 225 will be described more fully hereinafter. The catheter body 12 extends through a central bore in the proximal hub 225 and passes through a central lumen of the outer sheath 22. A second hub member 227, as depicted in FIGS. 16A and 16B, is coupled to a proximal end of the catheter body 12. The second hub member 227 and the first hub member 225 are removably engageable with each other as will also be described further hereinafter.

Depending upon the orientation of the filter member 16, the first end 18 or the second end 20 may either be fixed or moveable relative to the catheter body 12. Alternatively, as will be discussed further hereinafter, the filter member 16 may have only a first end 18 which is fixed to the catheter body 12.

In some embodiments, the catheter body 12 may have only a single lumen, rather than being a multi-lumen catheter.

To facilitate percutaneous introduction of the inventive filter catheter 10, a physician may optionally elect to employ an introducer sheath (not shown) as vascular access conduit for the filter catheter 10. The presence of the filter member 16 at the distal end of the catheter body 12 creates a region of relatively lower flexibility and the practitioner may determine it beneficial to employ an introducer sheath for vascular access.

As used in this application, unless otherwise specifically stated, the terms “proximal” and “distal” are intended to refer to positions relative to the longitudinal axis of the catheter body 12. Those skilled in the art will understand that the catheter body 12 has a distal end which is first inserted into the patient and a proximal end which opposite the distal end. Additionally, the terms “inferior” or “inferiorly” are intended to refer to the anatomic orientation of being in a direction away from the patient's head while the terms “superior” or “superiorly” are intended to refer to the anatomic orientation of being toward the patient's head.

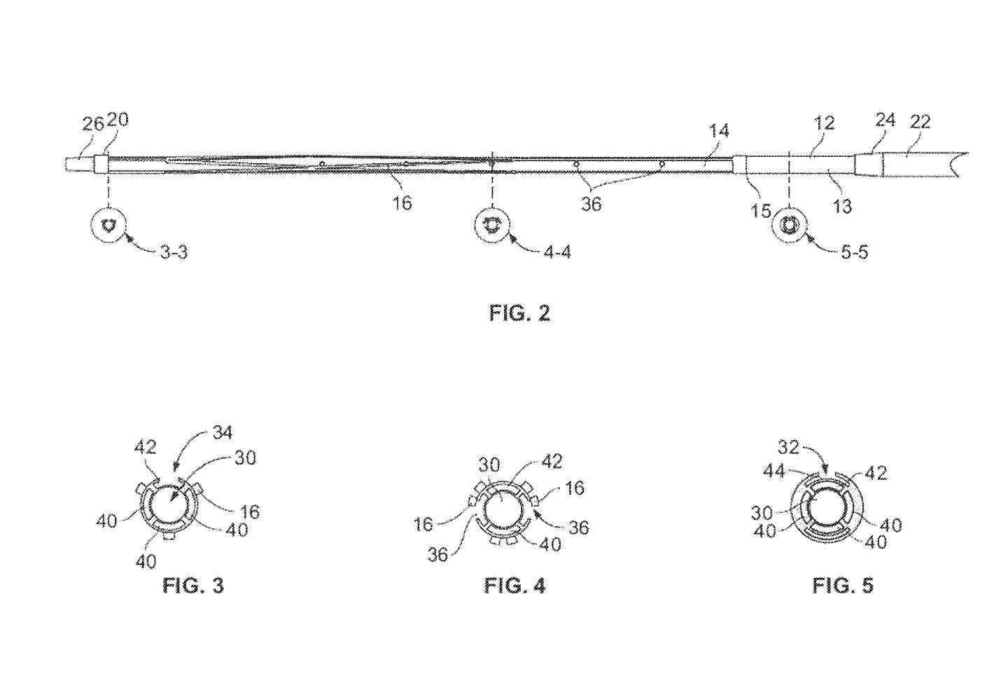

The multi-lumen aspect of the filter catheter 10 is shown more clearly in FIGS. 2-5. The catheter body 12 has a proximal section 13 and a distal section 14, which is longitudinally opposite the proximal section 13 and which may have a relatively smaller diametric profile than the proximal section 13. As described above, the first lumen 44 terminates at the proximal port 32, while the second lumen 42 terminates at the distal port 34. A central guidewire lumen 30 may be provided that extends the entire longitudinal length of the catheter body 12 and terminates at the distal end of the catheter body 12 at a distal guidewire opening 31 that permits the catheter body to track along a guidewire during a procedure. The central guidewire lumen 30 may also be used to introduce fluids, such as bioactive agents, intravenous fluids or blood transfusions.

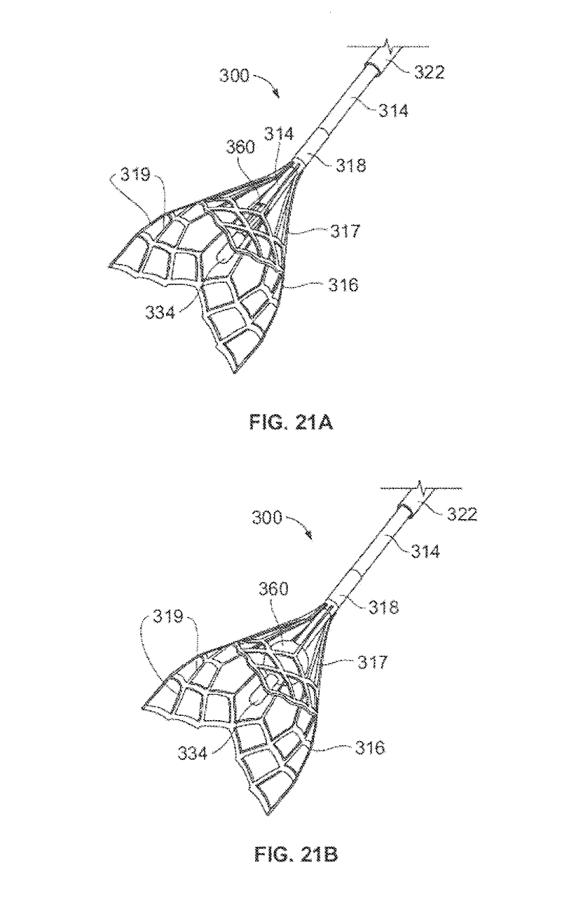

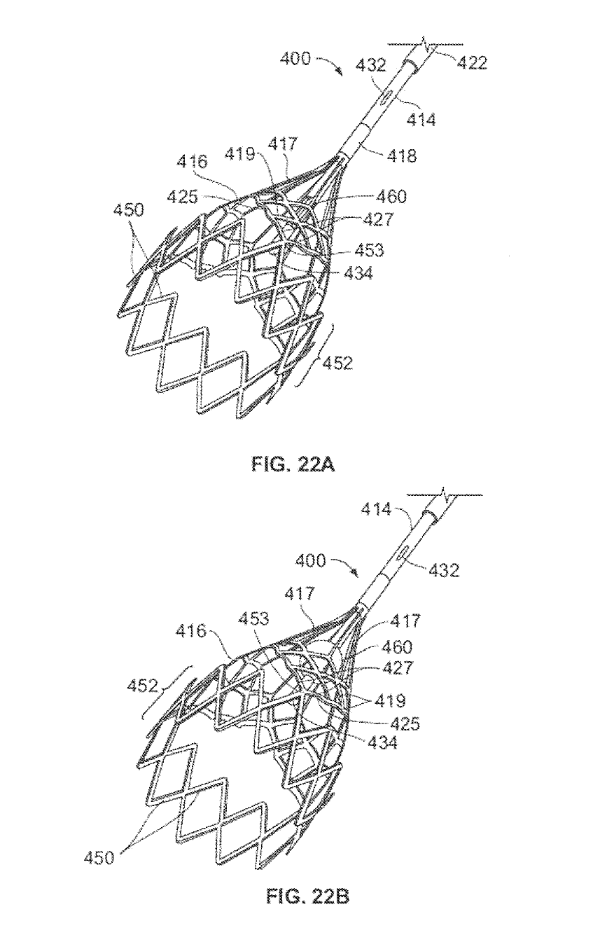

Additionally, at least one of a plurality of infusion lumens 40 are provided, each having at least one infusion port 36 that passes through a wall of the catheter body 12. Bioactive agents, flushing fluids for flushing or under elevated pressures for mechanical thrombolysis of thrombus in the filter member 16, contrast agents or other fluids may be infused through the infusion lumens 40 and out of the at least one infusion port 36 to pass into the patient's venous system for either local or systemic effect. In accordance with one embodiment of the invention, plural infusion ports 36 are provided with multiple ports 36 being provided in communication with a single infusion lumen 40 and spaced along a longitudinal axis of the catheter body 12. Additionally, plural infusion ports 36 may be provided in a circumferentially spaced manner to provide for fluid infusion at points spaced around the circumference of the catheter body 12. In this manner, fluid infusion is provided along both the longitudinal axis and the circumferential axis of the catheter body 12 within the spatial area defined by and bounded by the filter member 16. Because the plural infusion ports 36 communicate with the spatial area defined by and bounded by filter member 16, fluids introduced through the infusion lumens 40 are directed immediately at thrombus caught within the filter member 16. This permits thrombolytic agents or high pressure mechanical thrombolysis using a pressurized saline flush to be introduced directly to the situs of thrombus capture within filter member 16. Alternatively, thermal, ultrasound or other types of thrombolysis may be employed to disrupt thrombus captured by the filter member 16. For example, the annular space between the outer sheath 22 and the catheter body 12 may be used to introduce a thrombolytic to the filter and shower the filter to disrupt thrombus caught by the filter member 16. Additionally, the balloon depicted in FIGS. 21 and 22 may be positioned adjacent the filter member 16 and be provided with plural openings oriented in the direction of the filter member 16 to facilitate thrombolysis.

It will be understood, by those skilled in the art, that alternative arrangements of the first lumen 44, the second lumen 42, the guidewire lumen 30, or the infusion lumens are possible and contemplated by the present invention. The number and arrangement of lumens in the catheter body 12 is a function of the desired number of operable ports passing through the walls of the catheter body 12, the relative position of the operable ports, the desired position and geometry of the guidewire lumen 30, the desired longitudinal flexibility of the catheter body 12, the desirable degree of kink resistance of the catheter body 12, and other factors which are known to one of ordinary skill in the catheter arts.

While the present invention is not limited to specific dimensional sizes of either the catheter body member 12, the outer sheath 22, lumen diameter or port dimension, an exemplary outer diameter size of the outer sheath 22 is between 8 Fr (2.7 mm) and 9 Fr (3.0 mm) while an exemplary outer diameter size of the catheter member 12 is between 6 Fr (2.0 mm) and 7 Fr. A diametric transition taper 15 may be provided between the proximal portion 13 and the distal portion 14 of the catheter body 12 corresponding to the thickness of the filter member 16. In this manner, the outer surface of the filter member 16 is substantially co-planar with the outer diameter of the proximal portion 13 of the catheter body 12 about its entire circumference. Alternatively, the catheter body member 12 may have a constant diameter and the filter member 16 coupled to an outer surface of the catheter body member 12, with the outer sheath 22 having a luminal diameter sufficient to fit over the filter member 16. Moreover, the fixed first end 18 of filter 16 is positioned adjacent and in abutting relationship with the diametric transition 15, while the moveable second end 20 of filter member 16 is concentrically positioned around the distal section 14 of catheter body 12 and is reciprocally moveable thereupon to accommodate diametric expansion of the filter member 16. Lumen diameter and port dimension are a function of design requirements and are variable depending upon the desired purpose and function of the lumen or port, e.g., pressure sensing, infusion, evacuation, guidewire, flow sensing, or flow conduit.

In order to aid a physician in visualizing the filter catheter 10 in vivo, at least one radio-opaque or other viewable marker may be provided. A first marker 24 is provided at the distal end of the outer sheath 22 and a second marker 36 may be provided at a distal tip 33 of the catheter body 12. It will be understood that when the outer sheath 22 is in its non-retracted delivery position, that the filter 16 will be covered and the marker 24 and the second marker 36 will be adjacent or in close proximity with one another. Alternatively, the outer sheath 22 may, itself, be made of or include a radio-opaque or other viewable material, such as a metal braid or metal reinforcement within or applied to a polymeric sheath. The first and second markers 24, 36 or the material of the outer sheath 22 may enhance visualization of the filter catheter 10 under fluoroscopy, ultrasound or other visualization or guidance technique.

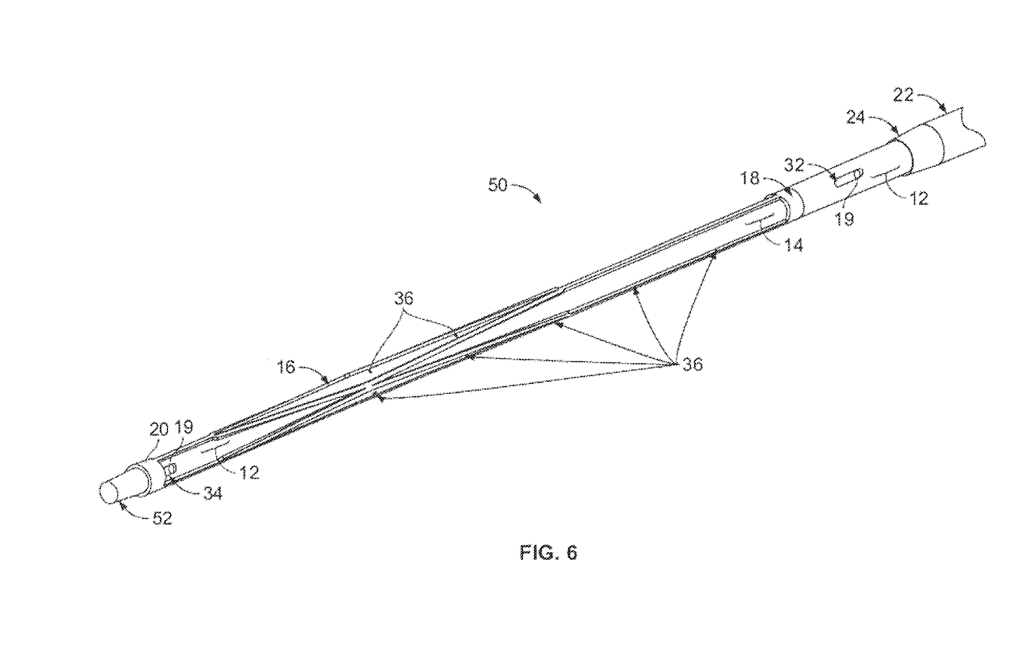

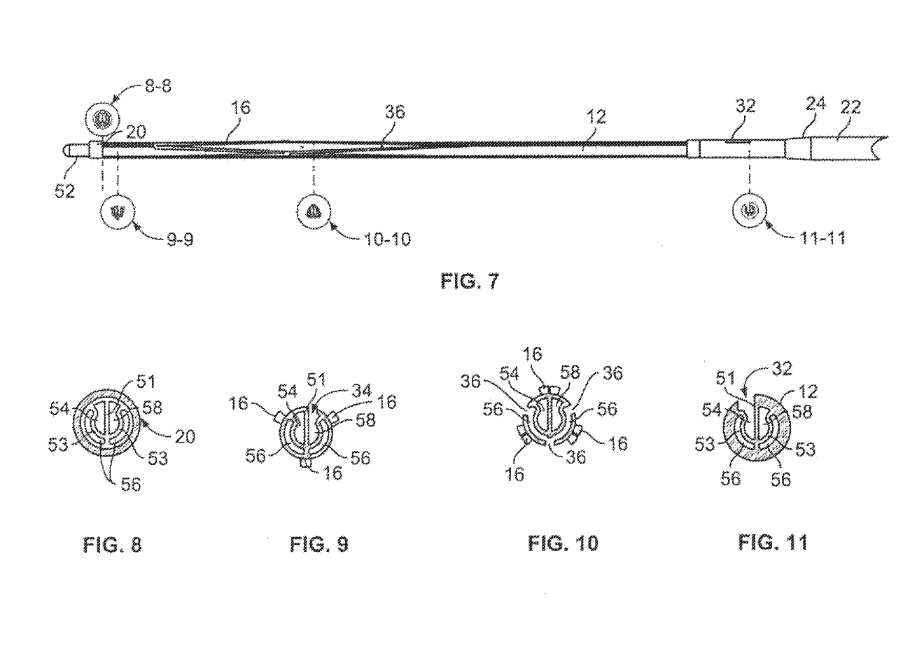

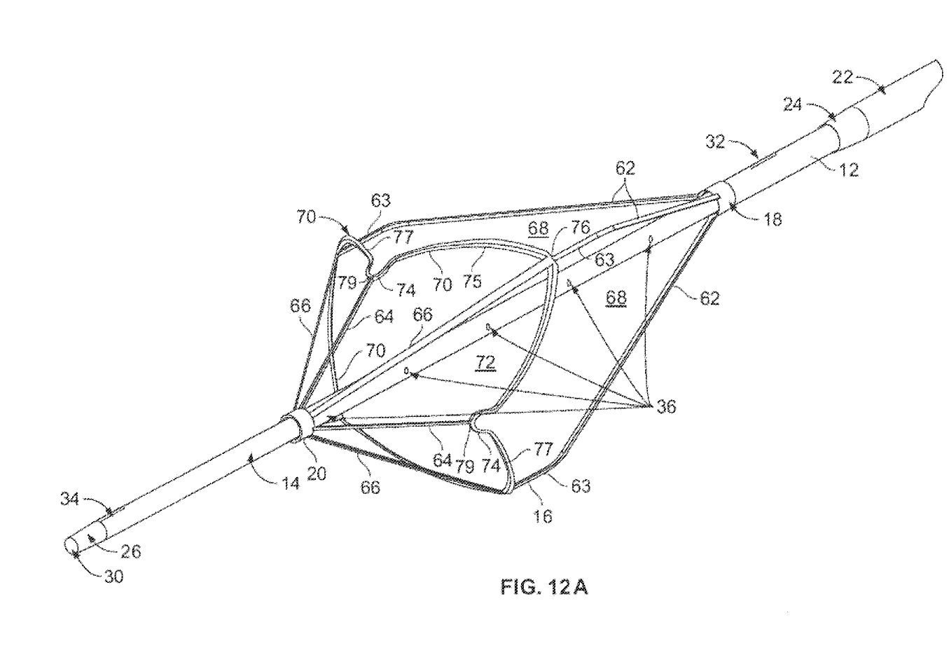

FIGS. 6-11 illustrate a second embodiment of the filter catheter 50. Unlike filter catheter 10, filter catheter 50 does not include the central guidewire lumen 30 of filter catheter 10. Rather, while the general construct of filter catheter 50 is similar to that of filter catheter 10, a different configuration of the inner lumens is employed.

Filter catheter 50, like filter catheter 10, consists generally of a multi-lumen catheter body 12 having a proximal port 32 associated with a first lumen 54 and a distal port 34 associated with a second lumen 58, a filter member 16, having a fixed proximal end 18 and a moveable distal end 20, is positioned generally intermediate the distal port 34 and the proximal port 32 and is generally concentric relative to the catheter body 12. Use of the term “generally intermediate” is intended to mean that at least a substantial portion of the filter member 16 resides intermediate the distal port 34 and the proximal port 32. Thus, the filter member 16 may partially overlay either or both of the proximal port 32 or the distal port 34.

The catheter body 12 has a proximal section 13 and distal section 14 which has a relatively smaller diametric profile than the proximal section 13. As described above, the first lumen 54 terminates at the proximal port 32, while the second lumen 58 terminates at the distal port 34. An atraumatic tip 52 terminates the catheter body 12 at its distal end. The atraumatic tip 52 preferably includes a radio-opaque marker to aid in positional visualization of the distal end of the catheter body 12.

A plurality of infusion lumens 56 are provided, each having at least one infusion port 36, preferably plural infusion ports 36, that passes through a wall of the catheter body 12 and communicates with a space defined within an area bounded by the filter member 16. Bioactive agents, flushing fluids, pressurized mechanical thrombolytic fluids, or other fluids may be infused through the infusion lumens 56 and out of the at least one infusion port 36 to pass into the space defined by the filter member 16 and ultimately into the patient's venous system for either local or systemic effect. In accordance with one embodiment of the invention, the each of the plural infusion lumens 56 are in fluid communication with plural ports 36 arrayed along both the longitudinal axis and the circumferential axis of the catheter body. This configuration provides for fluid infusion along both the longitudinal axis and the circumferential axis of the catheter body 12 and in direct communication with the space defined by the filter member 16 that captures thrombus.

The infusion lumens 56, the first lumen 54 and the second lumen 58 are bounded by and separated from each other by first catheter septum 51 and second catheter septum 56 which also aid in providing structural support for the catheter body 12. First catheter septum 51 is a generally diametrically and longitudinally extending member that divides the first lumen 54 from the second lumen 58 along the longitudinal axis of the catheter body 12. Second catheter septum 56 may comprise a generally U-shaped member that intersects the first catheter septum 51 at a lower aspect of the septum and is connected with an inner wall surface of the catheter body 12 at upper aspects of the septum 51 to define two infusion lumens in lateral regions of the catheter body 12.

The filter member 16 has two general configurations. A first configuration consists generally of two opposing generally open conical sections formed by plural interconnected structural elements defining the lateral surfaces of each open conical section, wherein the two opposing generally open conical sections each have open bases facing each other which are interconnected by a generally cylindrical section of the filter member 16. Each open conical section has an open base and an apex, wherein the apices project in opposing directions, with one apex projecting proximally and another apex projecting distally relative to the axis of the catheter. The plural interconnected structural elements forming the lateral surfaces of each generally open conical sections may be strut-like structural members extending generally axially along the longitudinal axis of the filter member 16. The axially extending strut-like structural members may be linear members or may be curved members. The apices of each of the generally open conical sections are formed either of a generally cylindrical collar that serves to couple the filter member 16 to the catheter body 12. The generally cylindrical collar is concentrically engaged about the catheter body 12 and may be axially movable thereupon, or is formed by connections between adjacent pairs of longitudinal strut-like structural members which circumscribe a circumference of the catheter body 12. The generally cylindrical section of the filter member 16 is formed by a generally open lattice of interconnected structural elements which connect the base of a first open conical section to the base of a second open conical section. The generally cylindrical section of the filter member 16 lies in apposition with a vascular wall upon deployment of the filter member 16 with a vascular lumen.

A second general configuration of the filter member 16 consists generally of a single generally open conical section in which a plurality of longitudinal strut-like structural members form the lateral surfaces of the conical section and are connected to a generally cylindrical collar which couples the filter member 16 to the catheter body 12 at an apex of the generally open conical section. The base of the generally open conical section is formed by opposing ends of the longitudinal strut-like structural members. A generally cylindrical section of the filter member 16, formed of a generally open lattice of interconnected structural elements, extends from the longitudinal strut-like structural members forming the base of the generally open conical section, to provide a region of the filter member 16 which is in apposition to the vascular wall upon deployment of the filter member.

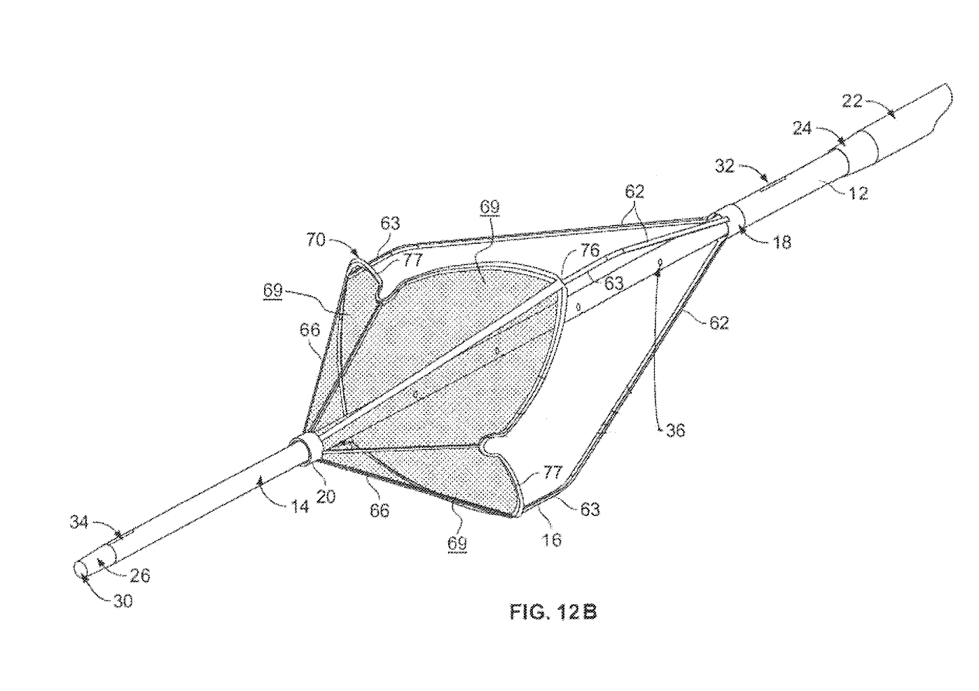

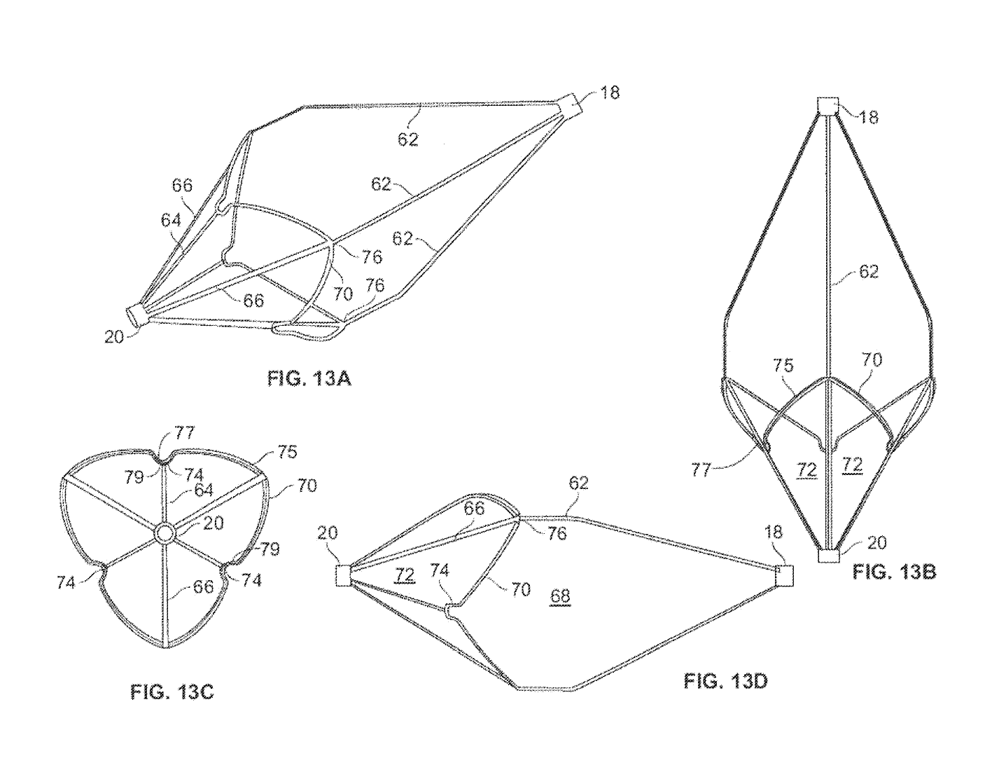

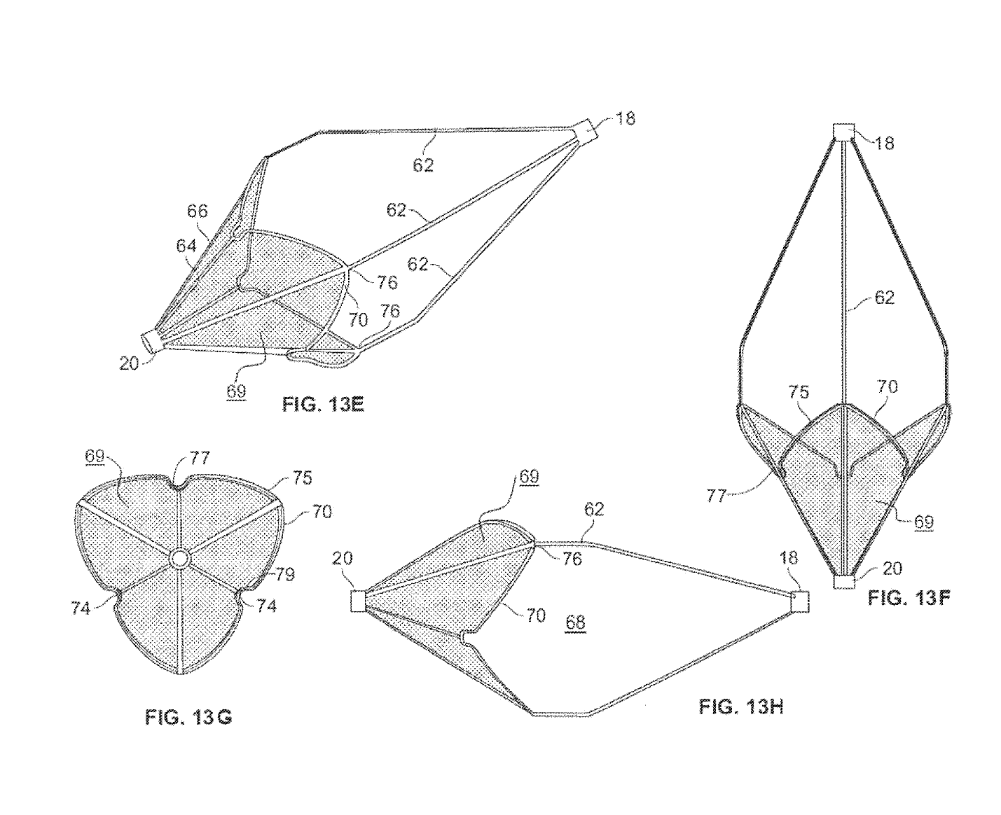

One embodiment of the filter member 16 is illustrated in its diametrically expanded configuration in FIGS. 12A-13H. In this embodiment, filter member 16 consists generally of a proximal end 18 and a distal end 20, each of which consists generally of a tubular ring-like structure which is circumferentially positioned about a section of the catheter body 12. One of the first end 18 and second end 20 are fixedly coupled to the catheter body 12, while the other is movable relative to the catheter body 12. At least one of a plurality of first strut members 62, preferably three, are coupled at their proximal end to the proximal end 18 of filter member 16 and each extends distally relative to the longitudinal axis of the catheter body 12. Each of the first strut members 62 is an elongate member that, upon diametric expansion of the filter member 16, flares away from the central longitudinal axis of the catheter body 12 and terminates in a distal end section 63 that bends distally and is generally parallel with the longitudinal axis of the catheter body 12. A plurality of second strut members 64, preferably three, are coupled at their distal end to a the distal end 20 of filter member 16 and each extends proximally relative to the longitudinal axis of the catheter body 12. A plurality of third strut members 66, preferably three, are coupled at their distal ends to the distal end of the filter member 16 and each extends proximally relative to the longitudinal axis of the catheter body 12. It will be appreciated, by those skilled in the art, that the number of struts employed as the first strut members 62, the second strut members 64 and the third strut members 66 forming the filter member 16 may be evenly distributed about a 360 degree circumference and define the lateral wall surfaces of the filter member 16.

A hoop member 70 extends circumferentially to define a circumferential axis of the filter member 16 and has a series of continuous undulations defining a series of peaks 77 and valleys 75 about the circumference of filter member 16. Each of the plurality of first strut members 62, the plurality of second strut members 64 and the plurality of third strut members 66 are coupled to the hoop member 70 at different points about its circumferential axis and intermediate the proximal end 18 and the distal end 20 of the filter member 16. In its unexpanded state the filter member 16 has a generally tubular shape, while in its expanded state the filter member 16 assumes one of the general configurations discussed above, i.e., either oppositely extending generally open conical sections or a single generally open conical section.

The plurality of first strut members 62 are preferably offset from each other by approximately 120 degrees about the circumference of the catheter body 12. The plurality of second strut members 64 are also preferably offset from each other by approximately 120 degrees. Finally, the plurality of third strut members 66 are also preferably offset from each other by approximately 120 degrees. Each of the plurality of first strut members 62 couple at a junction 76 to the hoop member 70 at a peak thereof. Similarly, each of the plurality of third strut members 66 couple at junction 76 to the hoop member 70 at a peak thereof. In this manner, a first strut member 62 and a third strut member 66 are each coupled to hoop member 70 at junction 76 and, in this relationship, form a generally linear member that extends along the longitudinal axis of the catheter body 12 and connects between the proximal end 18 of the filter member 16 and the distal end 20 of the filter member 16. Each of the second strut members 64 couple at their proximal ends to a valley 77 of the hoop member 70 and connects at a junction 79. Unlike the connections at junction 76 between the plurality of first strut members 62 and the plurality of third strut members 66, in this embodiment of the filter member 16, there is no member that connects to junction 79 and extends from the proximal end 18 of the filter member 16. In this configuration, the hoop member 70 assumes a generally circumferential tri-leaflet ring having three peaks 75 and three valleys 77.

The configuration of the struts of the filter member 16 may define a first cone 68 and a second cone 72. The first cone 68 is defined by the plurality of first strut members 62 between the proximal end 18 of the filter member 16 and the hoop member 70. The second cone 72 is defined by the plurality of second strut members 64 and the plurality of third strut members 66 between the distal end 20 of the filter member 16 and the hoop member 70. The first cone 68, formed by the first plurality of struts 62, may be configured so as to permit blood flow therethrough, and function as a frame for the second cone, which performs the function of filtering the emboli. The second cone 72 may be configured so as to permit blood flow therethrough, but such that thrombi and embolic material are captured by the pluralities of second and third strut members 64, 66. To further enhance the filtering capabilities of the filter member 16, the regions of the second cone 72 between the second strut members 64, third strut members 66, and the hoop member 70 may be covered by a cover member 69, as shown in FIGS. 12B and 13E-H. In some embodiments, the cover member 69 may comprise high density ePTFE.

Because of its biocompatible properties many types of surgical implants and prostheses have been made of polytetrafluoroethylene (PTFE). Successful vascular grafts are formed from expanded polytetrafluoroethylene (ePTFE), which is characterized by a microporous structure consisting of “nodes” interconnected by “fibrils.” Expanded PTFE tubular products are formed by admixing PTFE resin particles with an extrusion lubricant (e.g. mineral spirits) to form a slurry. This slurry is then compacted into a cylindrical extrusion billet, which is placed in a ram extruder and extruded through a die to form a tubular extrudate. The tubular extrudate is then dried, i.e., heated to evaporate the lubricant. As might be expected from material formed from compacted resin particles, the resulting extrudate has rather limited longitudinal and radial tensile strength. Expanded PTFE has a microscopic structure of nodes interconnected by fibrils and is normally not very porous. One measure of porosity is dimensional, e.g. 8-10 microns, or 0.1-100 microns, or 0.01-1000 microns. Unlike most other polymers, for PTFE this dimension is not the diameter of a hole or pore through the sheet but is the distance from one node to another among a plurality of nodes making up a pore. Since the nodes are interconnected by fibers, the dimension is a measure of fiber length. Porosity may be varied to achieve optimum flow through the cover member without disrupting blood flow in the vena cava or other veins or arteries in which the filter member is disposed. Porosity may also be measured as the percentage of pores occupying the area of the cover member. In one embodiment, the porosity may be between 50% and 99%, alternatively, between 60%-89%, alternatively between 70%-79% to achieve the optimum flow through. The porosity may be increased further by stretching the cover member. A stretch ratio may be selected for the cover member to adjust the flow rate in a particular vein or artery. For example, the stretching ratio of the cover member may be between 1.5 and 10 based on the area of the cover member stretched in the longitudinal direction, the transverse direction, or both directions to the area of the cover member in its unstretched state. Flow rates are discussed below, but the cover member may include a porosity to achieve a flow rate between 1.0-1000 mL/cm or for a blood entry pressure of 0-350 psi selected to a specific vein or artery.

While specific reference is made to using ePTFE as the biocompatible material for the cover member 69, alternative materials may be used, including polyamides, polyimides, silicones, polyethylene (PE); polypropylene (PP); Polyvinylidene Fluoride (PVDF); Ethyl Vinyl Acetate (EVA); fluoroethylpolypropylene (FEP), polypropylfluorinated amines (PFA), or other fluorinated polymers; Polycarbonate (PC) and many PC alloys such as PC/ABS (acrylontitrile-butadiene styrene); Thermoplastic polyurethane (TPU); Polyethersulfone (PES); composite materials; or other porous metals. Alternatively, the biocompatible material for the cover member may include, but is not limited to: 1) Treatment of alternate materials to achieve desired porosity (e.g.: laser machining); or 2) Construction of cover (e.g.: weaving of stranded material).

In some embodiments, the cover member 69 may comprise a single layer of ePTFE that is disposed over the exterior of the second/distal cone 72 of the filter member 16.

In some other embodiments, the cover member 69 may comprise a plurality of layers of ePTFE that are disposed over the interior and exterior of the second/distal cone 72 of the filter member 16, and then sintered together within the interstitial spaces between strut members. In this manner, the filter member 16 may be understood as having ePTFE layers on both the luminal and abluminal surfaces of the second/distal cone 72.

Alternatively, the layers of ePTFE to may be attached to both surfaces or sides of the filter member 16 by means other than applying pressure and sintering, such as applying an adhesive, an aqueous dispersion of PTFE, a PTFE tape, FEP, or a tetrafluoroethylene between the layers of PTFE and the filter member 16 and then heating the assembly to melting temperature below the sintering temperature of the PTFE layers.

In some further embodiments, at least one drug or bioactive compound may be loaded into the cover member 69, such as between the nodes of an ePTFE cover member 69.

The ePTFE cover preferably comprises initial internodal distances (INDs) within a range of 10 to 90 microns. Further, in some embodiments, the inner and outer ePTFE layers which comprise the covered filter member may have different INDs.

To facilitate bending and folding of the hoop member 70 between the expanded and unexpanded states, generally U-shaped hinge members 74 may be provided at each of the valleys 77 of the hoop member 70. It will be understood that each of the plurality of first strut members 62, plurality of second strut members 64, plurality of third strut members 66 and the hoop member 70 are preferably fabricated of biocompatible materials, such as shape memory alloys, superelastic materials or elastic materials, including, without limitation, titanium, vanadium, aluminum, nickel, tantalum, zirconium, chromium, silver, gold, silicon, magnesium, niobium, scandium, platinum, cobalt, palladium, manganese, molybdenum and alloys thereof, such as zirconium-titanium-tantalum alloys, cobalt-chromium-molybdenum alloys, nitinol, and stainless steel.

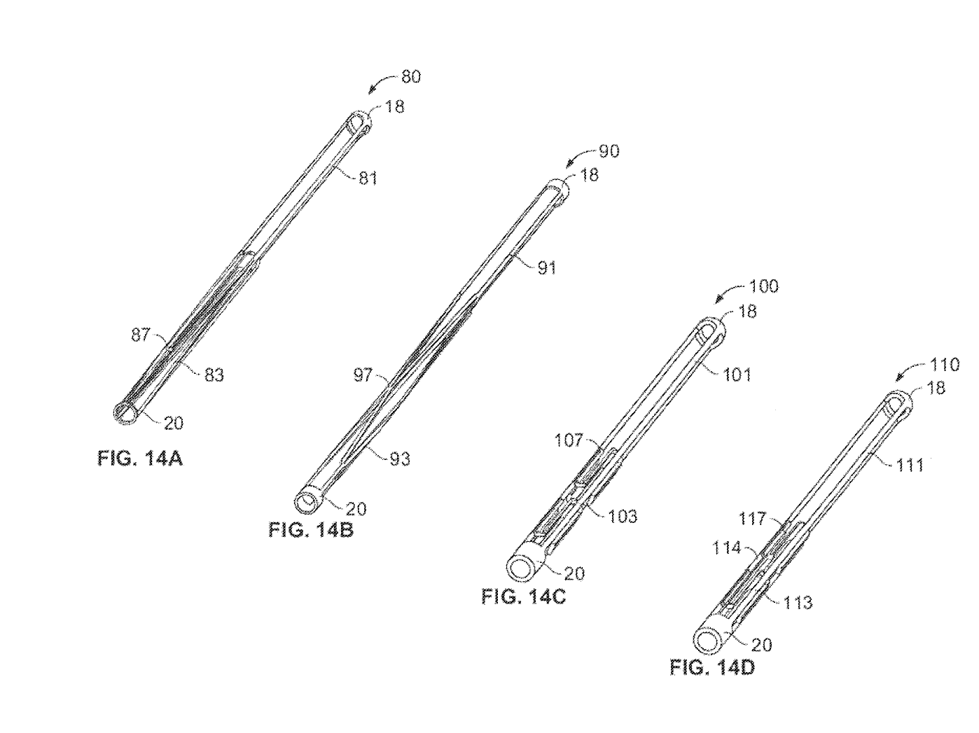

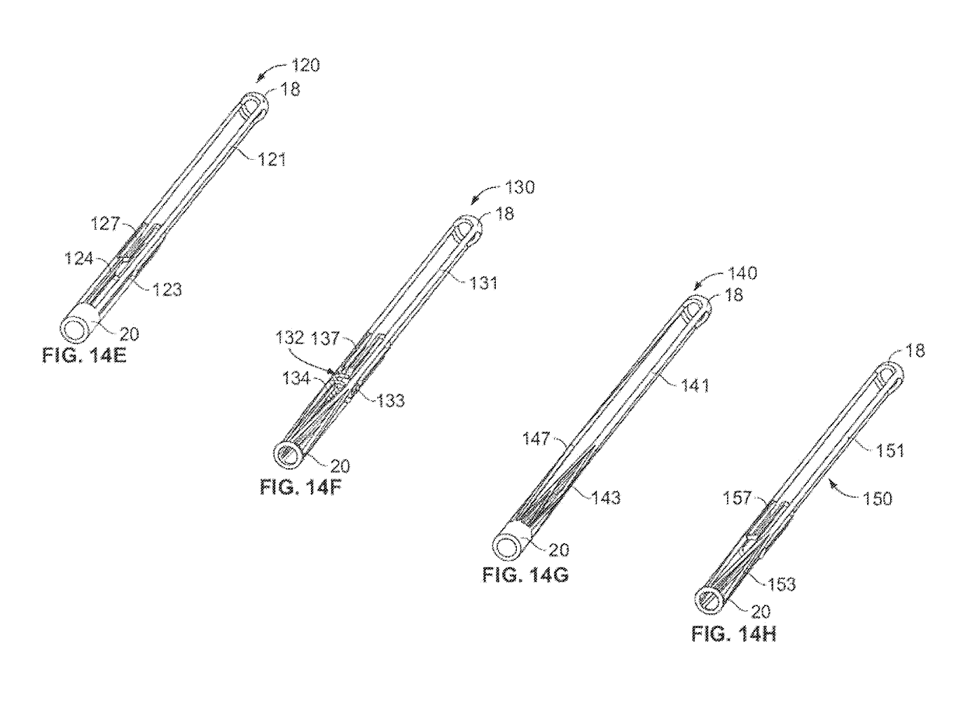

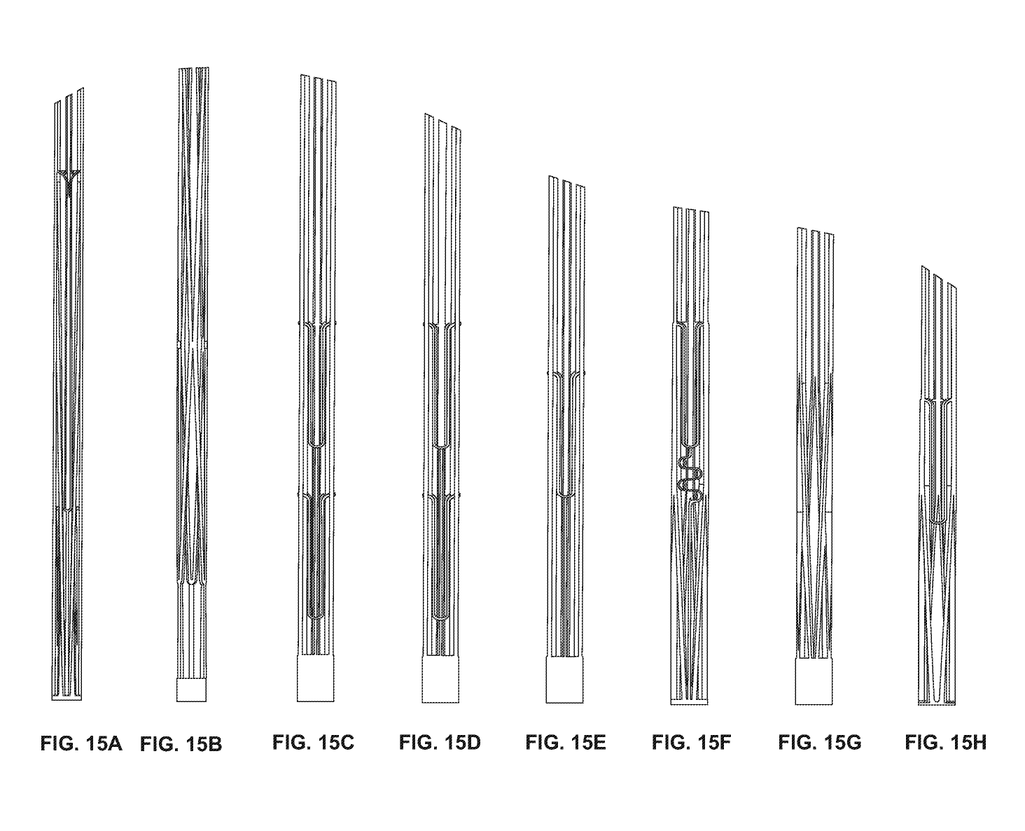

FIGS. 14A-14H and corresponding FIGS. 15A-15H depict alternative embodiments of the filter member 16, labeled 80, 90, 100, 110, 120, 130, 140 and 150, respectively. Like filter member 16, each of filter members 80, 90, 100, 110, 120, 130, 140 and 150 having a proximal end 18 and a distal end 20 that each consist of a generally ring-like structure intended to circumferentially couple to a catheter body 12 (not shown), with the proximal end 18 being fixed and the distal end 20 being reciprocally moveable axially along the distal portion 14 of catheter body 12. Like filter member 16, each of the alternative filter member embodiments depicted in FIGS. 14A-14H and 15A-15H, consist of a plurality of first strut members 81, 92, 101, 111, 121, 131, 141 and 151, respectively, extending distally from the proximal end 18 of the filter member and a plurality of second strut members 83, 93, 103, 113, 123, 133, 143 and 153, respectively, extending proximally from the distal end 20 of the filter member, with a diametrically expansible hoop member 87, 97, 107, 117, 127, 137, 147, 157, respectively, interconnecting the distally extending strut members 81, 92, 101, 111, 121, 131, 141 and 151, respectively, with the proximally extending strut members 83, 93, 103, 113, 123, 133, 143 and 153. In the alternative embodiments of filter members 100, 110 and 120, at least some distally extending strut members and at least some of the proximally extending strut members form linear elements that extend along the entire longitudinal axis of the respective filter member, with the hoop member being comprised of at least one undulating or serpentine ring structure.

In the alternative embodiments of filter members 80, 90, 130, 140 and 150, a plurality of distally extending strut members are provided spaced approximately 120 degrees apart from one and other about the circumference of the filter members, and the distally extending strut members bifurcating once or twice distally in a generally Y-shaped manner as in filter members 80, 130, 140 or 150, or the proximally extending strut members bifurcating proximally in a generally Y-shaped manner and interconnecting with the distally extending generally Y-shaped strut members to form a diamond-like pattern as in filter member 90. In filter members 90 and 140, the hoop member is formed by the diamond-like pattern formed by the intersection of the plurality of struts. In contrast, in filter members 80, 130 and 150, the hoop member is formed by at least one undulating or serpentine ring structure which is diametrically expansible. As illustrated in filter members 110, 120 and 130, apical portions of each undulating or serpentine ring structure is interconnected by an interconnecting member 114, 124, 134, respectively, either with an adjacent ring structure, as in filter member 110 or to a distal end 20 of the filter member itself. A longitudinally serpentine section 132 in filter 32 may be provided in conjunction with the interconnecting member 134, to afford greater expansive properties to the hoop member 137.

According to some embodiments particularly well-suited for placement by femoral or other infrarenal approach, the filter member 16 is characterized by a generally conical filter member 16 having a greater open surface area exposed to the flow of embolic material into the filter at its proximal end, while the distal end has smaller open surface area exposed to the flow of embolic material to capture the embolic material in the distal end of the filter member.

In other embodiments particularly well-suited for placement by a jugular or suprarenal approach, the filter member 16 is characterized by a generally conical filter member 16 having a greater open surface area exposed to the flow of embolic material into the filter at its distal end, which the proximal end of the filter member 16 has a smaller open surface area exposed to the flow to capture smaller embolic material in the distal end of the filter member 16.

Additionally, in all of the embodiments the filter member 16 is self-centering to provide proper apposition against the vascular walls and centering within the lumen of a blood vessel. This maximizes the flow dynamics of the filter member 16 within the blood vessel for purposes of capturing embolic material within the struts of the filter and centers the catheter body member 12 within the vascular lumen.

As noted above, the proximal 32 and distal 34 ports serve as means for measuring flow rates or pressure differentials across the filter 16. This may be accomplished by including flow sensors and/or pressure transducers 19 in operable association with each port 32, 34, with the associated electrical connections to the flow sensors and/or pressure transducers 19 passing through the respective lumens associated with each port 32, 34 and terminating at the proximal end of the catheter body 12. Where flow sensors 19 are employed, a single flow sensor associated with proximal port 32, the distal port 34 or the distal end of sheath 22 may be sufficient to detect fluid flow rate at the position of the catheter body 12. By providing a flow sensor at the distal end of sheath 22, the clinician will be able to determine flow velocity at the distal end of the introducer sheath 22 prior to introducing the catheter body 12 and make fine adjustments to the placement of the distal end of the introducer sheath 22 to ensure proper placement for the filter member 16. Plural flow sensors 19 may be employed and operably associated with each of proximal port 32 and distal port 34 to sense changes in flow velocity across the filter member 16. Alternatively, the flow sensors and/or pressure transducers 19 may reside in communication with the lumens respectively associated with each port 32, 34 at the proximal end of the catheter body 12, thereby eliminating the need for electrical connectors resident with the associated lumens. Furthermore, wireless flow sensors and/or pressure transducers may be provided in communication with each port 32, 34, and be operably coupled to a power source and a transmitter to wirelessly transmit telemetry data from the transducers to a wireless receiver in communication with the transmitter, as is known in the art.

Alternatively, the proximal 32 and distal ports 34 may be used for monitoring or sensing other conditions in the body that are detectable in the blood. For example, analyte sensors may be introduced to either the lumens communicating with the proximal 32 or distal ports 34 or to the ports themselves to monitor and/or sense chemical or biochemical conditions in the body. An example of this application is monitoring or sampling blood glucose levels for diabetes control. Further, the proximal 32 and distal ports 34 may be used for fluid infusion or for withdrawal or evacuation of fluids or other material through the catheter body 12. In this later instance, where the proximal port 32 is positioned to underlay the filter member 16, thrombus collected in the filter member 16 may capable of being lysed, either by thrombolysis through the infusion ports 36 or under the influence of thermal or mechanical lysis, such as by introducing a laser, ultrasound or other system capable of lysing thrombus, which may be introduced through the lumen communicating with the proximal port 32, or the distal port 32 or the guidewire lumen 30, or introduced separately from the filter catheter 10, positioned within the space bounded by the filter member 16, lysing thrombus collected in the filter member 16 and evacuating the lysed thrombus through the proximal port 32.

It is known that flow rate increases proximally within the venous system. For example a flow rate of 1 L/min is typical in one femoral vein, increases to 2 L/min in the inferior vena cava and increasing another 0.7 to 1 L/min proximate the renal veins. Knowing the typical flow velocities in vessels of different transverse cross-sectional areas, coupled with a flow sensor 19 associated with the multi-lumen catheter body 12 may serve to supplement or replace the requirements for fluoroscopy or sonography in placement of the filter catheter 10, 50.

Other sensors, such as, for example, chemosensors, color sensors, electrical sensors or biosensors, may be employed in lieu of or in addition to pressure transducer and/or a flow sensor 19 in order to detect other changes or conditions within the patient's vasculature. For example, color sensors exist that sense color changes in thrombus, such color changes may be displayed and interpreted by the medical practitioner as an indication of thrombus staging. Analyte sensors, such a as a glucose sensor or an oxygen saturation sensor may also be employed.

In some embodiments, the filter member 16, or its alternative embodiments described above, may be fixed to the catheter body 12 or may be removably coupled to the catheter body 12 for deployment as a temporary and retrievable filter. In some embodiments, the filter may be a vena cava filter. Removable coupling of the filter member to the catheter body 12 may be accomplished with a variety of release and retrieval mechanisms operably associated the catheter body 12 and proximate the diametric transition 15. Non-limiting examples of such release and retrieval mechanisms include a wire release that engages with a the proximal end 18 of the filter, a cooperating indexed detent and projection interaction between the catheter body 12 and the proximal end 18 of the filter, such as a detent in the proximal end of the filter and a cooperating projection in the multi-lumen catheter that is positionally indexed to the detent and releasable from the detent, or, alternatively, a helical slot or threads may be formed in the proximal end 18 of the filter and indexed and cooperating projection in the multi-lumen catheter than permits engagement and disengagement with the helical slot or threads.

In use, an introducer sheath is first placed into the body in a normal manner for introducing a central venous line, such as by the Seldinger technique. Specifically, after accessing a vein using a large bore needle, under local anesthesia, a guidewire is inserted through the needle bore and passed into the vein. Once the guidewire is positioned, the needle is withdrawn, and a dilator together with the introducer sheath introduced over the guidewire. Once the introducer sheath is positioned at a desired location within the venous system under radiography, the dilator may be removed from the patient. Radiopaque markers associated with the introducer sheath may be employed to assist in positional visualization of the distal end of the introducer sheath. The outer sheath 22 covering the filter 16 is removed while introducing the filter member 16 and catheter body 12 into the introducer sheath. The outer sheath 22 constrains the filter member 16 during its passage through the introducer sheath and positioning the distal end of the catheter within the patient's vasculature. Once the distal end of the catheter body 12 reaches the distal end of the introducer sheath, the filter is deployed. If the filter therapy alone is desired, the filter member 16 is detached from the catheter body 12 and the catheter body 12, introducer sheath and guidewire is withdrawn from the patient. Where both central venous access and filter therapy is desired, the introducer sheath and catheter body 12 with the filter member 16 is left in the patient until withdrawal is required.

Retrieval and removal of a detached filter member 16 is accomplished using a second procedure under local anesthesia which substantially replicates the placement of the filter catheter, with a capture sheath (not shown), similar to introducer sheath, being introduced, a retrieval catheter being introduced through the sheath, and engaging the filter member 16, then withdrawn into the capture sheath to collapse the filter member 16, with the entire assembly of the filter member 16, catheter body 12, outer sheath 22 and guidewire, if used, is withdrawn from the patient.

FIGS. 16A and 16B depict the undeployed state (FIG. 16A) and the deployed state (FIG. 16B) of a filter member 216 on an example implementation of a filter catheter 200. The filter catheter 200 includes an inner catheter 214 that carries the filter 216 at a distal end thereof. The inner catheter 214 is concentrically and reciprocally engaged within an outer sheath 222 such that relative axial movement of the inner catheter 214 and the outer sheath 222 either exposes the filter 216 for deployment or captures the filter 216 for retrieval. A first hub member 225 is coupled to a proximal end of the outer sheath 222 and a second hub member 227 is coupled to a proximal end of the inner catheter 214. First hub member 225 and second hub member 227 are engageable, such as by a threaded, bayonet, snap fit, friction fit or interference fit fitting, to secure the inner catheter 214 within the outer sheath 222 and restrict relative axial movement of the two elements after deployment of the filter 216. A flush line 229 communicates with the first hub member 225 and is in fluid communication with a luminal space within the outer sheath 222. A plurality of fluid lines 231, 233 communicate with the second hub member 227 and are each in fluid communication with one of the multiple lumens within the inner catheter member 214, e.g., lumens communicating with the proximal, distal or infusion ports (not shown). A distal tip 26 is provided at a distal end of the inner catheter.

In some instances, a jugular approach or other approach necessitates that the catheter be introduced retrograde relative to the vector of blood flow within the vessel, i.e., the catheter is introduced through the jugular vein and directed inferiorly toward an infrarenal position. Additionally, since the blood flow opposes the distal end of the catheter and passes toward the proximal end, the filter must open inferiorly such that its largest diametric section in apposition to the vessel walls opens toward the distal end of the catheter rather than toward the proximal end of the catheter as with the femoral approach.

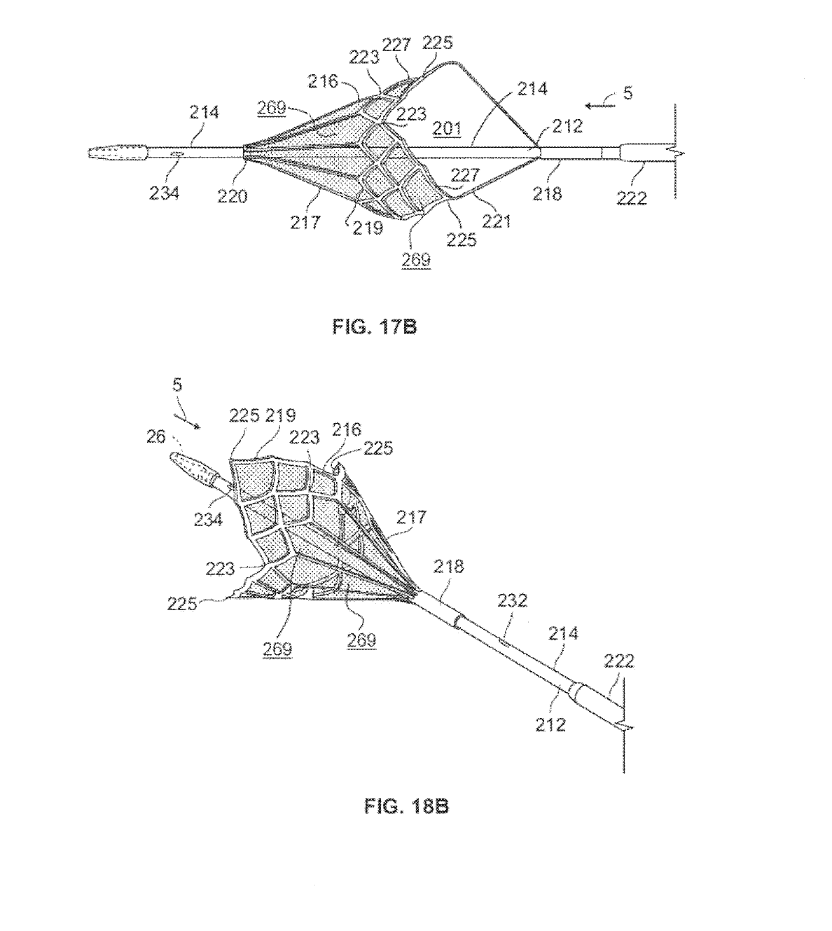

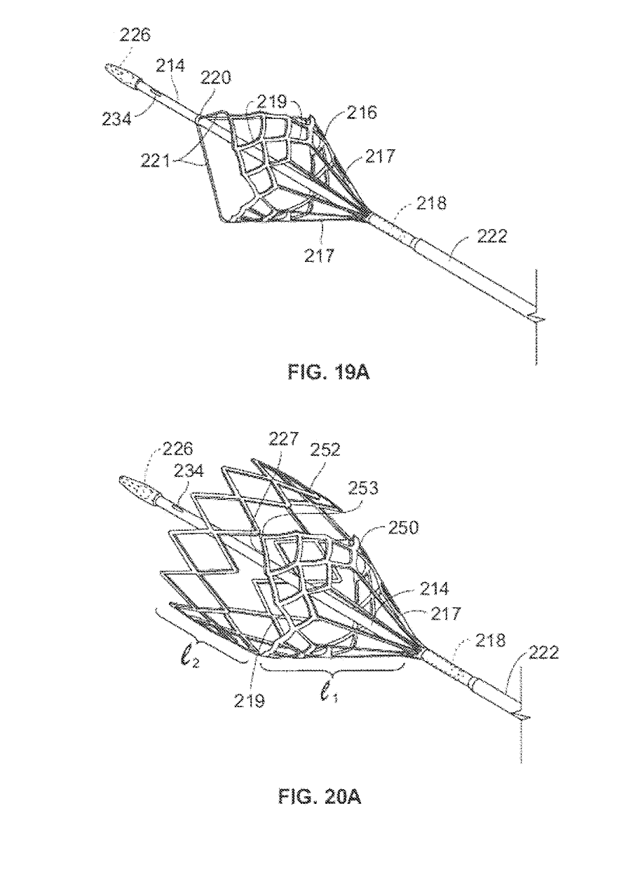

FIGS. 17A-20B depict alternative embodiments of filter members in accordance with the present invention. FIGS. 17A-B illustrate a filter orientation for a femoral approach, while FIGS. 18A-20B illustrate a filter orientation for a jugular approach. As illustrated in FIGS. 17A-B, filter member 216 defines a relatively larger volume open space 201 and a relatively smaller volume open space 203. Open spaces 201 and 203 are bounded by structural members of the filter member 216 and are both open toward the direction of blood flow indicated by arrow 5, with larger open space 201 being relatively upstream the blood flow relative to smaller open space 203 in both the femoral or the jugular orientation of filter member 216. FIGS. 17B, 18B, 19B, and 20B each depict the same embodiment as FIGS. 17A, 18A,19A, and 20A, respectively, with the addition of a cover member 269, as discussed above. In FIGS. 17B and 18B, the cover member 269 is illustrated as disposed over the structural members 217, 219 of the filter member 216 that bound open space 203.

In some embodiments, the cover member 269 may comprise a single layer of ePTFE that is disposed over the exterior of the structural members 217, 219 of the filter member 216 that bound the open space 203.

In some other embodiments, the cover member 269 may comprise a plurality of layers of ePTFE that are disposed over the interior and exterior of the structural members 217, 219 that bound the open space 203 of the filter member 216, and then sintered together within the interstitial spaces between strut members. In this manner, the filter member 216 may be understood as having ePTFE layers on both the luminal and abluminal surfaces of the open space 203.

Alternatively, the layers of ePTFE to may be attached to both surfaces or sides of the filter member 216 by means other than applying pressure and sintering, such as applying an adhesive, an aqueous dispersion of PTFE, a PTFE tape, FEP, or a tetrafluoroethylene between the layers of PTFE and the filter member 216 and then heating the assembly to melting temperature below the sintering temperature of the PTFE layers.

As with all previous embodiments described of the filter member, filter member 216 is formed of plural interconnected structural elements. In accordance with the preferred embodiments of the filter members of the present invention, and as particularly exemplified by filter member 216, the filter member has a first end 218 and a second end 220, at least one of which is attached to the distal section 214 of the catheter body 212. First structural members 217 extend generally axially, either proximally as shown in FIGS. 17A-B or distally as shown in FIGS. 18A-B, along the longitudinal axis of the filter member 216. Again, it is understood that use of the terms “proximal” or “proximally” and “distal” or “distally” are intended to refer to positions relative to the longitudinal axis of the catheter body 212. The first structural members 217 are connected to either the first end 218 or the second end 220 of the filter member 216. Second structural members 219 are connected to the first structural members 217 at an end of the first structural members 217 which is opposite that connected to either the first end 218 or the second end 220 of the filter member 216. In accordance with a preferred embodiment of the invention, the second structural members 219 form at least two successive zigzag shaped structures which are connected to an end of the first structural members and at opposing apices 223 to form conjoined ring-like structures about the circumference of the filter member 216. In this manner the second structural members 219 generally define lattice-like pattern upon diametric expansion of the filter member 216. The lattice-like pattern formed by the second structural members 219 projects axially along the longitudinal axis of the catheter 214 tapering to form at least one petal-like projection 225 that terminates in a terminal apex member 227. As will be appreciated by those skilled in the art, FIGS. 17A-B depict three petal like projections 225, with one being behind the plane of the figure and, therefore, not shown. Each of the petal-like projections 225 act to engage and oppose vascular wall surfaces to seat the filter member 216 against the vessel wall, and center the filter member and catheter 214 within the vascular lumen. As illustrated in FIGS. 17A-B, third structural members 221 are provided and are connected to each of the terminal apex members 227 and extend axially relative to the catheter 214 and connect with a second end 218 of the filter member 216.

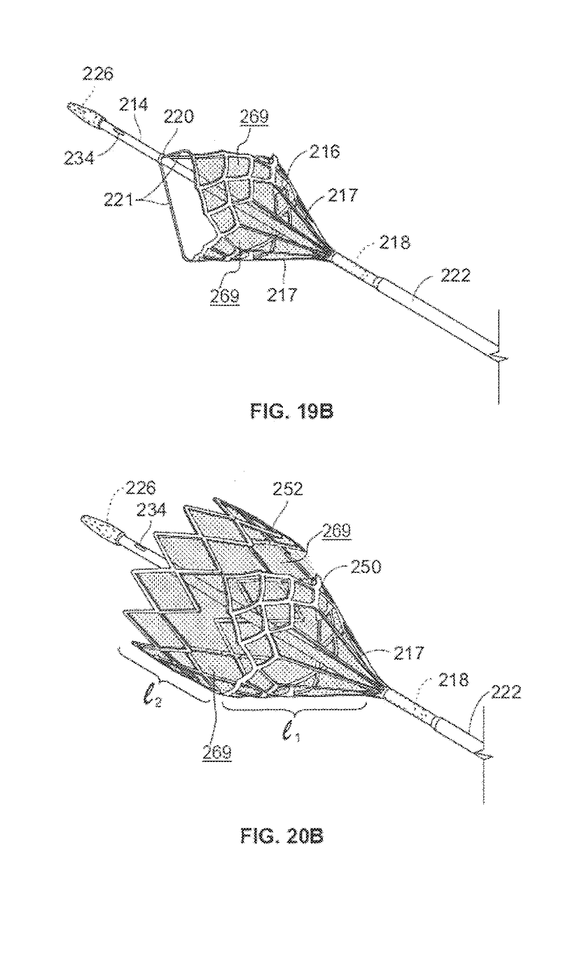

In the embodiment illustrated in FIGS. 17A-B, which is an orientation of the filter member 216 for a femoral approach, and in the embodiment illustrated in FIGS. 19A-B, which is an orientation of the filter member 216 for a jugular approach, the first end 218 of the filter member 216 is fixedly connected to the catheter 212, while the second end 220 of the filter member 216 is movably coupled to the catheter 212 and moves axially along the catheter 216 upon expansion or contraction of the filter member 216.

FIGS. 18A-B depict an embodiment of the filter member 216 identical to that illustrated in FIGS. 19A-B, with the sole exception that the third structural members 219 and the second end 220 of the filter member 216 are omitted. In this embodiment, the terminal apex member 227 of each petal-like member 225 are not connected to a second end 220 of the filter member 216 by the third structural members 219.

FIGS. 20A-B depict an alternative embodiment of the filter member 216 which is similar to that depicted in FIGS. 18A-B, except that at least one circumferential ring member 252 is connected to the terminal apex member 227 of each of the petal-like members 225 at a juncture 253 with the terminal apex member 227. The addition of the additional circumferential ring member 252 results in a relative elongation over the length L1 of the filter member 216 depicted in FIGS. 18A-B by a length L2 which facilitates additional apposition between the filter member 216 and the vascular wall and stabilization of the petal-like members 225.

FIGS. 21A and 21B depict an alternative embodiment of the filter member 216 in FIG. 18A, having first end 318, first structural elements 317 and second structural elements 319 all analogously arranged as in the embodiment of FIG. 18A. Filter member 300, however, employs a modified distal end 314 of the catheter 312 to include an expansive balloon 360. The guidewire lumen of the multi-lumen catheter 312 may be used in place of a distal port for condition sensing, flushing, infusion or the like. The expansive balloon 360 may be used to break up thrombus captured within the filter member 316, either by mechanical force through serial dilatation or by infusion of a thrombolytic agent through openings in the balloon 360. FIG. 21A depicts the balloon 360 in its collapsed state, whereas FIG. 21B depicts the balloon in its expanded state.

Alternatively, an expansive balloon 360 may be placed proximal the filter member 300 and serve to temporarily occlude the vessel to facilitate aspiration or evacuation of thrombus from the filter member 30. The expansive balloon may further be combined with the cover member previously discussed to provide an occlusive functionality to the device of the present invention.

Finally, FIGS. 22A and 22B depict an alternative embodiment of the filter member 216 in FIG. 20A having first end 418, first structural elements 417 and second structural elements 419, at least one circumferential ring member 452 connected to the terminal apex member 427 of each of the petal-like members 425 at a juncture 453 with the terminal apex member 427; all analogously arranged as in the embodiment of FIG. 20A. Filter member 400, however, employs a modified distal end 414 of the catheter 412 to include an expansive balloon 460. The guidewire lumen of the multi-lumen catheter 412 may be used in place of a distal port for condition sensing, flushing, infusion or the like. The expansive balloon 460 may be used to break up thrombus captured within the filter member 416, either by mechanical force through serial dilatation or by infusion of a thrombolytic agent through openings in the balloon 460. FIG. 22A depicts the balloon 460 in its collapsed state, whereas FIG. 22B depicts the balloon in its expanded state.

Again, an expansive balloon 460 may be positioned proximal the filter member 416 to permit temporary occlusion of the blood vessel and permit aspiration or evacuation of thrombus from the filter member 416. The expansive balloon may further be combined with the cover member previously discussed to provide an occlusive functionality to the device of the present invention.

Alternatively, in any of the embodiments of the present invention, the high porosity ePTFE cover member may be configured to provide an occlusive capability to the filter member.

It will be appreciated by those skilled in the art that in all embodiments of the described filter catheter, the filter member has a relatively larger opening that is open inferiorly in a direction that opposes the blood flow vector and employs structural elements that taper superiorly along the direction of the blood flow vector to reduce the open surface area of the filter member and capture thrombus.

In further embodiments, the entire basket (i.e., one of the cones) of the filter member maybe replaced by a soft flexible netting structure that achieves porosity through openings separated by fibers. This soft, flexible netting structure forms the porous section in example implementations of the filter member and may be made of ePTFE, thermo polymers, PTFE, and/or the like. The remainder of the structure of the filter member may comprise suitable metallic or non-metallic materials, including but not limited to NiTi, CoCr, and/or the like. The soft filter basket may be fixated through an attached catheter or wire, or through fixation hooks.

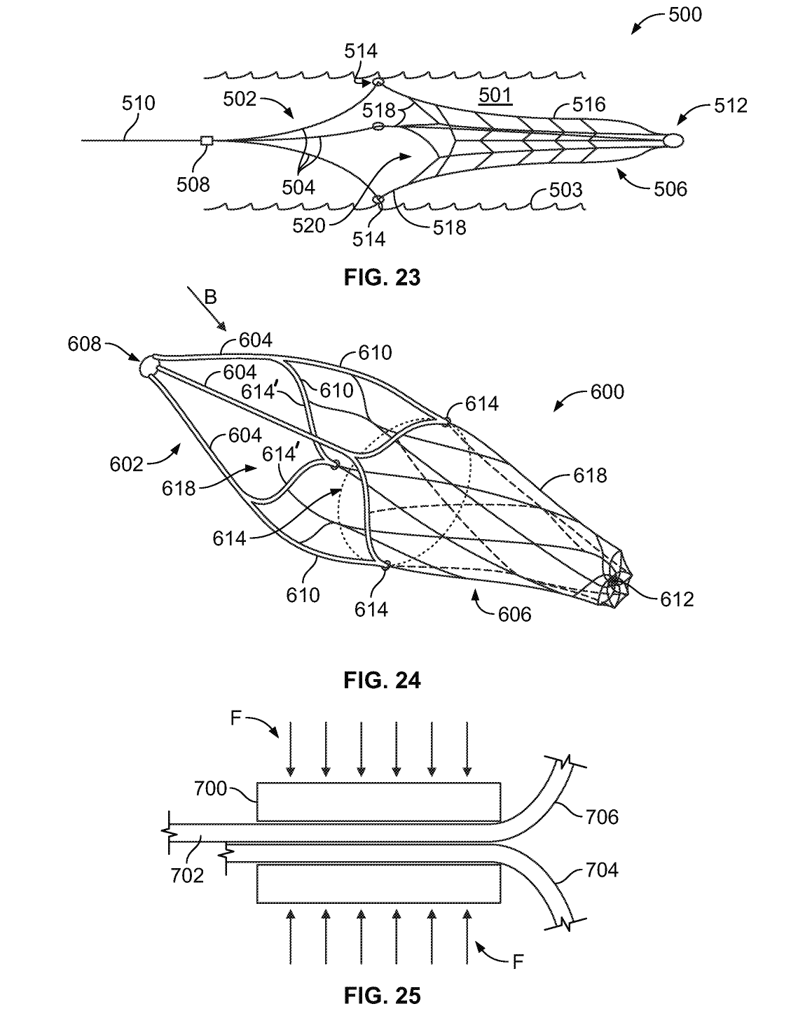

FIG. 23 is a side view of an example of a filter 500 for trapping thrombi in a blood vessel 501. The filter 500 includes a frame 502 formed by a plurality of frame members 504 extending from a frame end 508 to attach to a soft basket 506. The soft basket 506 is attached to the frame members 504 by a plurality of basket fixation elements 514. The soft basket 506 comprises a netting 516 surrounding an inner basket region 520. A plurality of basket attachment portions 518 are attached to corresponding basket fixation elements 514. The soft basket 506 includes a distal basket closure 512 to permit trapping thrombi in the inner basket region 520 when deployed in the blood vessel 501. The plurality of basket attachment portions 519 may include basket fixation elements 514 as described in more detail below with reference to FIGS. 25-27 for fixing the frame members 504 to an inner surface 503 of the blood vessel 501.

The filter 500 may be deployed as a standalone filter 500 that is pushed using a guidewire or catheter 510 through a sheath to a location in a patient's blood vessel 501. The guidewire 510 may then be detached from the filter 500 to leave the filter 500 at the location to capture any thrombic material passing through the blood vessel 501 at the location. The filter 500 may later be removed by extending the wire or catheter 510 through a catheter or sheath and hooking the wire to the frame end 508 before pulling the filter through the sheath or catheter. The filter 500 may also be attached to a catheter and deployed along with the catheter for a desired period of time without detaching the filter 500 from the catheter. In an example implementation, the filter 500 may be attached to a catheter of the types described above with reference to FIGS. 1-22.

The netting 516 of the soft basket 506 may be made of any suitable soft, flexible material that is woven or formed in a net-like structure having openings formed by strands, fibers, or string-like structures. Examples of materials that may be used for the netting 516 include ePTFE, thermo polymers including thermoset and thermoplastic polymers, PTFE, shape memory polymers (SMP), and/or the like. The openings in the netting are sized to capture large clinically significant thrombi while allowing adequate blood flow through the blood vessel 501.

The frame members 504 may be made of a material that is more rigid than that of the soft basket 506. For example, the frame members 504 may be made of nitinol, CoCr, or any other metallic or non-metallic material. In example implementations, the frame members 504 are made of a metallic or pseudo-metallic material having shape memory and flexibility so that the frame members 504 may be compressed but would return to an expanded state when compression forces are removed. In an example implementation, the frame members 504 may be constructed and attached to a catheter in a manner similar to that described above for the first strut members 62 in FIGS. 12A-12H where the first strut members 62 are modified for attachment of the soft basket 506.

FIG. 24 is a perspective view of another example of a filter 600 having a soft basket 606 attached to a frame 602. The soft basket 606 is formed of a netting 616 that encloses an inner basket region 618 bounded distally by a distal basket closure 612.

The frame 602 of the filter 600 in FIG. 24 comprises a plurality of frame members 604 that divide to form extended frame members 610. The extended frame members 610 branch out and connect to adjacent extended frame members 610 at a first plurality of basket fixation elements 614. The soft basket 606 may also attach at a second plurality of basket fixation elements 614′ at points of the extended frame members 610 between the point at which the extended frame members 610 branch out from the plurality of frame members 604 and the point where the extend frame members 610 connect to adjacent frame members 610. The frame 602 is configured so that openings formed by the plurality of frame members 604 and the plurality of extended frame members 610 permit blood flow towards the soft basket 606 as indicated by arrow B. The openings formed by the plurality of frame members 604 and extended frame members 610 are sufficiently large to permit thrombic material to pass through the frame 602 into the soft basket 606. The openings formed in the netting 616 of the soft basket 606 are small enough to trap the thrombi that are large enough to be clinically significant.

FIG. 25 is a side cross-sectional view of an example of a basket attachment tube element 700. The basket attachment tube element 700 is a basket fixation element for attaching the soft basket 606 (in FIG. 24) to the frame 602 (in FIG. 24). The basket attachment tube element 700 may be a tubular structure of any suitable material that may be crimped to hold whatever is inside the tubular structure. The frame 602 (in FIG. 24) includes an extending frame member 702 positioned inside the tubular structure of the basket attachment tube element 700. The soft basket 606 (in FIG. 24) includes a filament 704 extending from the netting 606 (in FIG. 24) into the inside of the tubular structure of the basket attachment tube element 700. The extending frame member 702 is inserted into one end of the basket fixation element 700 and the filament 704 of the soft basket is inserted into the opposite end of the basket fixation element 700. With the filament 704 of the soft basket and the extending frame member 702 of the frame in the inside of the tubular structure of the basket attachment tube element 700, the basket attachment tube element 700 may be crimped by forces F to bind the extending frame member 702 and the filament 704 together. The extending frame member 702 may be hooked at its end protruding from the basket attachment tube element 700 to provide a fixation portion 706. The fixation portion 706 on the extending frame member 700 advantageously provides a hooking structure that may be used to attach the extending frame member 702 to a blood vessel wall during deployment thereby fixing the opening of the soft basket 606 (in FIG. 24) to the wall of the blood vessel.

In an example implementation, the basket attachment tube element 700 in FIG. 25 may include a layer of, or may be made of, a material that is radio opaque. The basket attachment tube element 700 would then function as a radio-opaque marker that would appear in, for example, x-ray imaging.

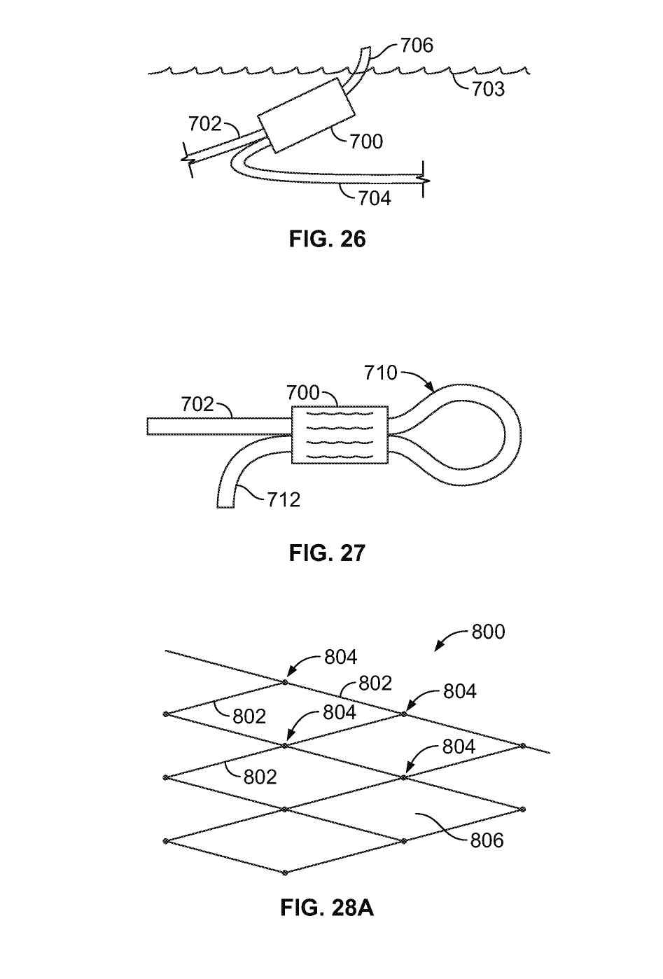

FIG. 26 is a side view of another example of a basket attachment tube element 700 with a fixation portion 706. In the example shown in FIG. 26, the extending frame member 702 and the filament 704 are inserted into the same end of the basket attachment tube element 700 before crimping. The fixation portion 706 extends from the opposite end of the basket attachment tube element 700 to hook to a blood vessel wall 703. The basket attachment tube element 700 may be a radio-opaque marker as described above with reference to FIG. 25.

FIG. 27 is a side view of another example of a basket attachment tube element 700 with a loop 710. The loop 710 is formed by the extending frame member 702 extending from the basket attachment tube element 700 and looping back into the basket attachment tube element 700. The extending frame member 702 continues to extend through the side of the basket fixation element 700 opposite the loop 710 to form a hook 712. The hook 712 advantageously affixes to the blood vessel wall during deployment. The soft basket 606 (in FIG. 24) may be attached by tying a filament (not shown in FIG. 27) of the soft basket 606 to the loop 710. The basket attachment tube element 700 may be a radio-opaque marker as described above with reference to FIG. 25.

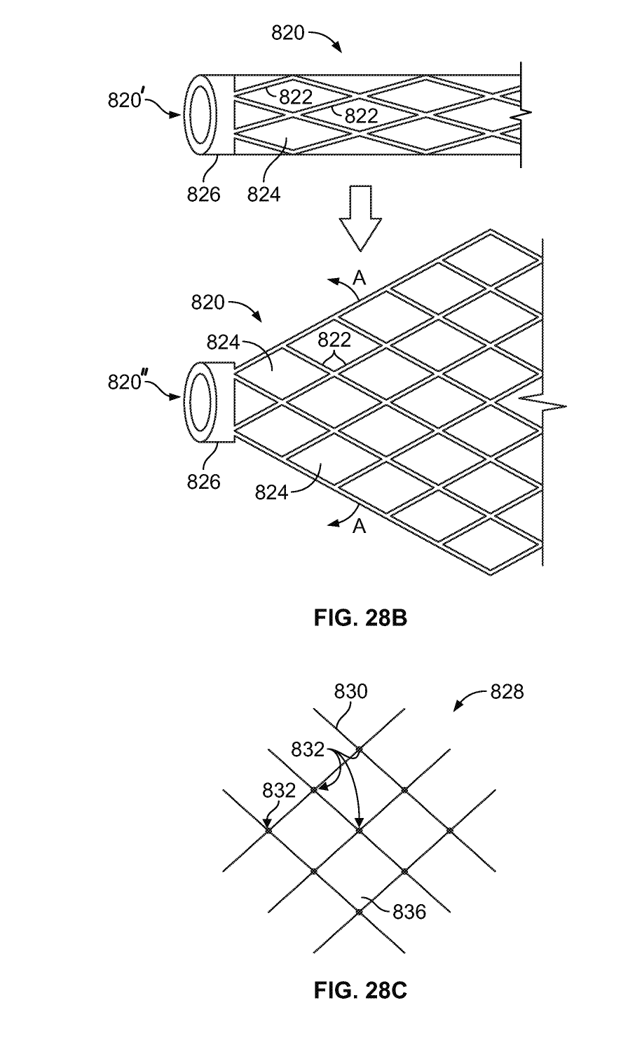

FIG. 28A is a top view of a section of an example implementation of netting 800 for a soft basket. The netting 800 comprises filaments 802 attached to one another at knots 804. The filaments 802 may be made of a pliable fiber, such as a polymer fiber, for example. The filaments 802 are attached with knots 804 that fix the filaments to one another at predetermined locations so as to form openings 806 in the netting of a size that is sufficiently small to trap clinically significant sized thrombic material.

FIG. 28B shows another example implementation of netting 820 for a soft basket. The netting 820 in FIG. 28B may be formed from a tubular structure 820′ with portions cutaway to form filaments 822 surrounding spaces 824. The tubular structure 820′ includes a tube end 826 that maintains a substantially tubular structure. The tubular structure 820′ may be expanded to form a netting structure 820″ as indicated by arrows A to expand the spaces 824 bounded the filaments 822 to a size that is sufficiently small to trap clinically significant sized thrombic material. The netting may be made of tube of flexible, elastic material that remains pliable after expansion.

FIG. 28C shows another example implementation of netting 828 for a soft basket. The netting 828 comprises filaments 830 attached to one another at reflow junctions 832. The filaments 830 may be made of a pliable fiber, such as a polymer fiber, for example. The filaments 830 are attached by a reflow process that bonds the filaments 830 to one another with a thermoplastic polymer at the reflow junctions 832. The reflow junctions 832 are positioned to form openings 836 in the netting of a size that is sufficiently small to trap clinically significant sized thrombic material. The reflow junctions 832 may be formed by reflowing the filaments 830, similar to, for example, spot welding metallic wires together. The reflow junctions 832 may also be formed by using a second material to mechanically bond the fibers. For example, a polymer bead may be melted to bond the filaments 830.

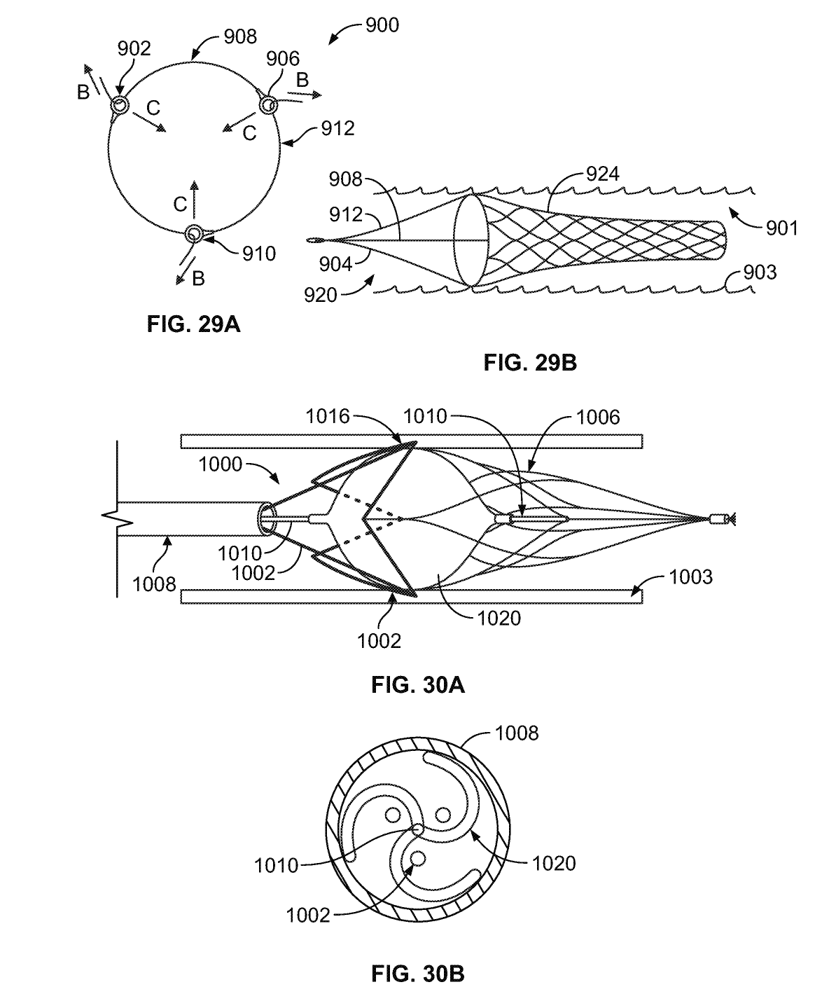

FIGS. 29A and 29B illustrate an example implementation of a filter 900 having a frame 920 and soft basket 924 that closes by pulling on the frame 922. The frame 920 includes frame members 904, 908, 912 threaded through corresponding loops 902, 906, 910. The first loop 902 is connected to a distal end of the first frame member 904. The second loop 906 is connected to the distal end of the second frame member 908. The third loop 910 is connected to the third frame member 912. Each frame member 904, 908, 912 is threaded through the loop 902, 906, 910 adjacent to it and extends proximally. The structure of the frame 920 is such that the soft basket 924 is closed (Arrows C) when a pulling force (Arrows B) is exerted on the frame members 904, 908, 912.

In the implementation illustrated in FIGS. 29A and 29B, the frame members 904, 908, 912 may be made of a shape memory material with a small diameter cross-section. The diameter of the cross-section of the frame members 904, 908, 912 should be sufficiently small so that the frame members 904, 908, 912 are sufficiently flexible to close the soft basket 924 when a pulling force is applied to the frame members 904, 908, 912. The frame members 904, 908, 912 should be configured with the shape memory material so that the frame members 904, 908, 912 expand to open the soft basket 924 when no force is applied to the frame members 904, 908, 912.

FIGS. 30A and 30B illustrate deployment of an example filter 1000 with a soft basket 1006 using a balloon catheter 1010. The filter 1000 in FIGS. 30A and 30B include a frame 1002 attached to the soft basket 1006. The frame 1002 may be attached to the balloon catheter 1010, which includes a balloon 1020 at a distal end of the balloon catheter 1010. The balloon catheter 1010, frame 1002, and soft basket 1006 are movably disposed in a lumen of a tubular catheter body 1008. The frame 1002 and balloon 1020 in its deflated state are compressible to fit in the lumen of the catheter body 1008 when the balloon catheter 1010 and frame 1002 are pulled into the lumen of the catheter body 1008.

FIG. 30B is a cross-sectional view of the catheter body 1008 showing the frame 1002, the balloon 1020 and balloon catheter 1010 collapsed within the catheter body 1008.

When the filter 1000 is positioned in a desired location of a patient's blood vessel 1001, the balloon catheter 1010 is pushed distally so that the frame 1002 and balloon 1020 in its deflated state exit a distal end of the catheter body 1008. The balloon 1020 may be inflated via a lumen in the balloon catheter 1010 thereby pushing the frame 1002 outwardly until a fixation portion 1016 in the basket fixation elements hooks into the blood vessel wall 1003. The balloon 1020 is then deflated and maintained in a deflated state while the filter 1000 performs its filtering function.

In an alternative embodiment, the frame 1002 may be attached to the inner surface of the catheter body 1008, or to another catheter structure that may be inserted into the catheter body 1008. The balloon catheter 1010 may also be removable allowing for the balloon 1020 to be deflated and the balloon catheter 1010 removed once the filter 1000 is deployed. In an embodiment in which the balloon 1020 is maintained in place as the filter 1000 performs its function, the balloon 1020 may be used to assist in thrombus mitigation. In an embodiment in which the balloon catheter 1010 is movable within the catheter body 1008, there is flexibility in how the balloon may be positioned to perform thrombus mitigation. In an embodiment in which the balloon and balloon catheter are removed, aspiration of thrombus through a lumen in the catheter body becomes another option for managing the thrombic material captured in the filter.

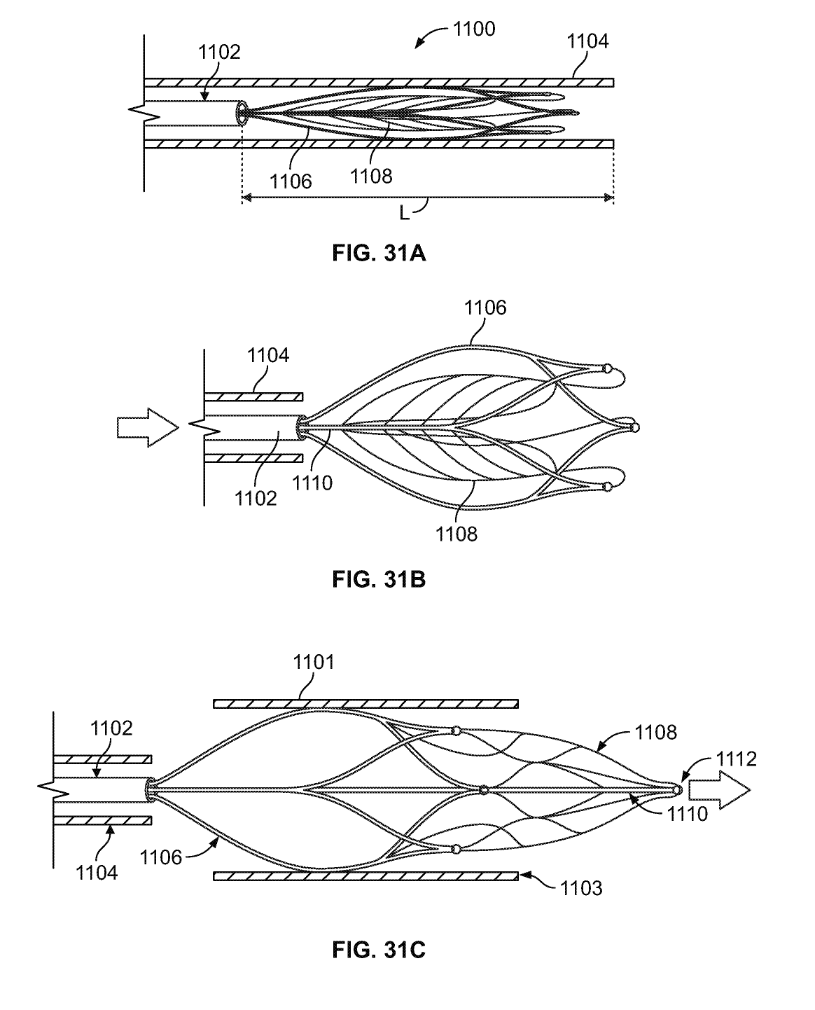

FIGS. 31A and 31B illustrate deployment of an example filter 1100 with a soft basket 1108 using a frame deployment member 1102 and a basket deployment member 1110. The filter 1100 comprises a frame 1106 and attached soft basket 1108. Prior to deployment, the frame 1106, soft basket 1108, and frame deployment member 1102 are collapsed and movably disposed within a distal end of a sheath 1104 with the soft basket 1108 inverted and disposed within the surrounding collapsed structure of the frame 1106. By inverting the soft basket 1108 in the structure of the frame 1106 when collapsed within the sheath 1104, the filter 1100 occupies about half the space within the sheath. The frame 1106 may be attached proximally to an inner wall of the frame deployment member 1102. The basket deployment member 1110 is movably disposed in a lumen of the frame deployment member 1102 (see FIG. 31B, not visible in FIG. 31A). The distal tip of the basket deployment member 1110 is attached to a distal closure 1112 of the soft basket 1108 (see FIG. 31C).

The filter 1100 is deployed by first pushing the frame deployment member 1102 distally within the sheath 1104 until the frame 1106 exits the distal end of the sheath 1104 as shown in FIG. 31B. As shown in FIG. 31B, the soft basket 1108 remains inverted within the structure of the frame 1106. The soft basket 1108 is then deployed by pushing the basket deployment member 1110 distally within the lumen of the frame deployment member 1102 to evert the soft basket 1108 into its operational form. The frame deployment member 1102 and the basket deployment member 1110 may be locked into a position in which the soft basket 1108 is in its operational form and the frame 1106 is in substantial contact with the blood vessel wall 1103 to maintain the filter 1100 in its functioning position as shown in FIG. 31C.

The foregoing description of implementations has been presented for purposes of illustration and description. It is not exhaustive and does not limit the claimed inventions to the precise form disclosed. Modifications and variations are possible in light of the above description or may be acquired from practicing the invention. The claims and their equivalents define the scope of the invention.

What is claimed is:

1. A filter for trapping thrombi in a blood vessel, the filter member comprising:
a frame formed by a plurality of frame members joined at a frame end, the frame members extending from the frame end to a filter attachment end; and
a filtering portion extending from the filter attachment end of the plurality of frame members, a least of portion of the filtering portion comprising a porous section made of a soft flexible material.

2. The filter of claim 1 where:
the frame members are formed by a plurality of first struts that form a first cone section having a plurality of proximal interstitial openings; and
the filtering portion comprises a second cone section formed by at least a plurality of second struts, the second struts configured to form a plurality of distal interstitial openings, where the porous section is a cover member disposed to cover at least one of the distal interstitial openings.

3. The filter of claim 2, wherein the cover member comprises layer of high porosity expanded polytetrafluoroethylene (“ePTFE”), polyamide, polyimide, silicone, fluoroethylpolypropylene (“FEP”), polypropylfluorinated amines (“PFA”), and fluorinated polymers.

4. The filter of claim 2, wherein the cover member has a porosity between 50% and 99% where porosity is measured as the percentage of pores occupying the area of the cover member.

5. The filter of claim 2 where the cover member is made of a stretched material that is stretched to a stretch ratio of between 1.5 and 10, where the stretch ratio is the area of the cover member in a stretched state over the area of the cover member in an unstretched state, and where the cover member is stretched in a longitudinal direction, a transverse direction, or both directions.

6. The filter of claim 1 where the filtering portion comprises a soft basket where the porous section comprises a netting forming the soft basket, where the netting surrounds an inner basket region, the soft basket comprising a plurality of basket attachment portions attached to corresponding basket fixation elements, and a distal basket closure.

7. The filter of claim 6 where the plurality of basket fixation elements are attachment tube elements each configured to contain a frame member end and a basket attachment portion end, where the attachment tube elements are crimped to join the frame member end to the basket attachment portion end.

8. The filter of claim 7 where the attachment tube elements are radio opaque markers.

9. The filter of claim 7 where the frame members are made of a rigid material, and where a distal end of each frame member is extended beyond the attachment tube element a length sufficient to provide a fixation hook to hook the frame member to the blood vessel wall.

10. The filter of claim 6 where:
the frame members are made of a rigid material,
a distal end of each frame member is looped back to form a loop with the looped back portion of the distal end of the frame member, and
the plurality of basket fixation elements include attachment tube elements each configured to contain the distal end of the frame member and the looped back portion so that the loop extends beyond the crimped attachment tube to attach to the soft basket netting.

11. The filter of claim 10 where the looped back portion of the distal end of the filter member extends beyond the crimped attachment tube on the side opposite the loop a distance sufficient to form a fixation hook to hook the frame member to the blood vessel wall.

12. The filter of claim 6 further comprising:
a filter deployment mechanism connected to the frame end of the frame.

13. The filter of claim 12 where the filter deployment mechanism is a releasable coupling mechanism configured to permit a releasable coupling to a guidewire, where the guidewire is used to push the filter into its deployment location, where the guidewire is detached from the filter for operation in the deployment location, and where the guidewire is releasably coupled to the filter for retrieval of the filter.

14. The filter of claim 12 where the filter deployment mechanism is a catheter, the frame end of the filter being attached to a distal end of the catheter.

15. The filter of claim 14 where the catheter comprises:
a tubular catheter body; and
the filter deployment mechanism further comprises a balloon catheter disposed in the tubular catheter body, the balloon catheter comprising a balloon attached to a distal end of the balloon catheter and a balloon deployment lumen extending proximally in the balloon catheter to a balloon deployment port configured to couple to a balloon fluid source for filling the balloon to spread the frame members outward until the frame members contact the blood vessel wall.

16. The filter of claim 14 further comprising:
a basket deployment member having a distal tip attached to the distal basket closure;
a frame deployment member having a frame deployment lumen, where the basket deployment member is disposed in the frame deployment lumen and is movable between a pre-deployed basket position in which the basket deployment member is retracted into the frame deployment lumen so as to invert and enclose the soft basket in the frame and a deployed basket position in which the basket deployment member extends beyond a distal end of the frame deployment member pushing the distal basket closure beyond the frame; and
a sheath having a sheath lumen, where the frame deployment member is disposed in the sheath and is movable between a pre-deployed frame position in which the frame is collapsed within the sheath lumen and a deployed frame position in which the frame is extended beyond a distal end of the sheath to expand to contact the blood vessel wall.

17. The filter of claim 6 where:
each of the plurality of basket attachment portions comprises a loop connected to a distal end of one of the plurality of frame members, and
each frame member is threaded through the loop connected to an adjacent one of the plurality of frame members such that a pulling force on the flexible members during retrieval of the filter operates to close the soft basket.
